(19) United States
(12) Patent Application Publication
(10) Pub. No.: US 2005/0197711 A1
(43) Pub. Date: Sep. 8, 2005

(54) Catheter deliverable foot implant and method of delivering the same

(76) Inventor: Victor V. Cachia, San Juan Capistrano, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/965,657

(22) Filed: Oct. 14, 2004

Related U.S. Application Data
(60) Provisional application No. 60/549,767, filed on Mar. 3, 2004.

(51) Int. Cl. A61F 2/42; A61F 2/46

(52) U.S. Cl. 623/21.11; 623/23.67; 623/23.7; 623/908

(57) ABSTRACT

Methods and devices are disclosed for manipulating alignment of the foot to treat patients with flat feet, posterior tibial tendon dysfunction and metatarsophalangeal joint dysfunction. An enlargeable implant is positioned in or about the sinus tarsi and/or first metatarsal-phalangeal joint of the foot. The implant is insertable by minimally invasive means and enlarged through a catheter or needle. Enlargement of the implant alters the range of motion in the subtalar or first metatarsal-phalangeal joint and changes the alignment of the foot or toe.

### CROSS-REFERENCE TO RELATED APPLICATIONS

The present application claims priority under 35 U.S.C. § 119(e) to U.S. Provisional Application No. 60/549,767 filed on Mar. 3, 2004, the disclosure of which are incorporated by reference herein in its entirety.

### BACKGROUND OF THE INVENTION

1. Field of the Invention

This invention relates generally to the field of subtalar joint and first metatarsal-phalangeal implants for treating foot conditions including flat feet, adult posterior tibial tendon dysfunction and metatarsophalangeal joint dysfunction.

2. Description of the Related Art

Pes valgo planus, or flat foot, is a common condition where the arch of a foot is weakened and is unable to properly support the weight of the body. With a flat foot, shock absorption is reduced and misalignment of the foot occurs. These changes may eventually result in foot and ankle pain, tendonitis, plantar fasciitis and hallux valgus, hallux limitus and functional disorders of the knees, hips and back. Although there are several causes of flat feet, one frequent cause is excessive motion in the subtalar joint of the foot.

As early as 1946, surgeons have been attempting to apply the arthroereisis concept to the subtalar joint. Arthroereisis is a surgical procedure for limiting motion in a joint in cases of excessive mobility. One early method was to remedy abnormal excursion of the talus on the calcaneus with the talus contacting the floor of the sinus tarsi by using an “abduction block” procedure. During the abduction block procedure, a wedge-shaped bone graft was impacted into the anterior leading edge of the posterior facet of the calcaneus. Impacting such a bone graft prevented excessive inferior displacement of the talus upon the calcaneus, thus limiting the amount of excess pronation of the subtalar joint.

A pronation limiting osteotomy in the form of a lateral opening wedge of the posterior facet was developed for treatment of “flatfoot” in cerebral palsy patients in 1964. In order to prevent interfering with subtalar joint motion, a wedge-like bone graft was used to improve the weight-bearing alignment of the calcaneus. In 1970, an accessory bone graft placed in the sinus tarsi was developed as a corrective procedure. Later, the bone graft was replaced with a silastic plug. As early as 1976, a high molecular weight polyethylene plug was developed. The plug is cemented into the calcaneal sulcus against a resected portion of the posterior calcaneal facet. This procedure, known as “STA-peg” (subtalar arthroereisis-peg), is a commonly used subtalar joint arthroereisis procedure. STA-peg does not block excessive pronation, but rather alters the axis of motion of the subtalar joint.

In addition, in 1976, a high molecular weight, polyethylene, threaded device known as a “Valenti Sinus Tarsi Arthroereises Device” was invented. The procedure used to implant the Valenti device is commonly referred to as the “Valenti” procedure. Unlike the STA-peg procedure, the Valenti procedure is an extra-articular procedure that involves placing the Valenti device into the sinus tarsi to block the anterior and inferior displacement of the talus. Such placement of the Valenti device does not restrict normal subtalar joint motion, but does block excessive pronation and resulting sequelae. The Valenti device has a frusto-conical shape and threads on the outer surface of the device, which allow it to be screwed into the sinus tarsi. Because of the shape of the Valenti device, the greater the penetration of the device into the sinus tarsi, the more the sinus is dilated and the more calcaneal eversion is eliminated.

However, several problems reduce the desirability of the Valenti procedure and device. Because of its frusto-conical shape and the manner in which it is inserted, the Valenti device is difficult to precisely position in the subtalar joint and difficult to ensure that the proper amount of calcaneal eversion has been eliminated. Furthermore, it is generally difficult to locate the device properly within the tarsal canal because the implant must be threaded at least 3 to 5 millimeters medial to the most lateral aspect of the posterior facet for correct placement. Because of its polyethylene construction, the device cannot be imaged using radiography (X-ray) to determine whether the proper position has been achieved.

More recent attempts to control subtalar motion in the hyperpronated foot include the Maxwell-Brancheau arthroereisis (MBA), the Kalix subtalar prosthesis and the Futura arthroereisis. The MBA is a titanium alloy implant where the implantation procedure involves insertion “trial” implants to determine the proper size of the actual implant used. The MBA implant procedure requires either general anesthesia or local anesthesia with sedation. It also requires up to a ¾ inch incision on the lateral portion of the foot. The MBA implant uses a metal guide pin for positioning the implant. The guide pin must be positioned with extreme care to prevent damage to the calcaneus. A two-week period of crutch use and foot immobilization typically follows the procedure. The Kalix implant is a cone-shaped implant with limited expansion ability. The operator can use a double screwdriver to increase the diameter of the implant. The Kalix implant requires two weeks of non-weight bearing and three to four weeks of immobilization following implantation of the device.

Another site of frequent foot problems is the first metatarsal-phalangeal joint. The first metatarsal-phalangeal joint (MTP) is a complex joint of the foot where bones, tendons and ligaments work together to transmit and distribute the body's weight, especially during movement. Bunions are the first MTP joint disorder most frequently treated by podiatric surgeons. First-line treatment involves educating patients about the condition and evaluating their footwear. Healthcare providers can direct their patients to wear wider, low-heeled shoes, use bunion pads, apply ice and take over-the-counter analgesic medications. These options are designed to relieve pain and make it easier to walk and engage in physical activities, but they do not address the underlying cause of bunions.

Bunions usually occur from inherited faulty biomechanics that put abnormal stress on the first MTP joint and medial column of the foot. Contrary to popular belief, bunions are aggravated, not caused, by shoes. Various non-surgical approaches can help prevent aggravation of bunions and other MTP-related problems. For some patients, non-surgical treatment is sufficient, but surgical intervention is considered if the bunions are progressive or if non-operative treatments provide inadequate improvement.

Bunion surgery is performed to repair tendons and other soft tissue and remove a small amount of bone. Procedures to correct more severe bunions may involve removal of the bump or minor realignment of the big toe joint. The most severe and disabling bunions often require extensive joint realignment, reconstruction, implants or joint replacement. Significant morbidity and recuperation time is required for such procedures.

First MTP-related problems also occur from repetitive trauma to the area and from arthritis. Over time, active persons can put continuous stress on the first MTP joint that eventually wears out the cartilage and lead to the onset of arthritis. This condition, known as hallux rigidus, causes loss of movement and pain in the joint. In most situations, non-operative treatments can be prescribed to provide relief, but those with advanced disease might need surgery, especially when the protective covering of cartilage deteriorates, leaving the joint damaged and with decreased range of motion. Again, significant morbidity results from these procedures and an extended recovery time is required.

Notwithstanding the foregoing, there remains a need for improved devices for treating subtalar and first-MTP related foot conditions.

### SUMMARY OF THE INVENTION

In one embodiment of the invention, a radially-expandable subtalar joint implant is inserted percutaneously into the sinus tarsi. The implant is inserted percutaneously into the foot through an access which has a diameter smaller than the sinus tarsi. During insertion, the implant is maintained in a closed configuration, i.e. a first, reduced diameter. The implant is inserted with a delivery tool so that it extends through the sinus tarsi in the foot. When the implant is properly placed within the foot, the delivery tool is withdrawn.

Once in place, the implant expands radially outward, assuming an open configuration, i.e. a second, expanded diameter, and anchoring itself in place. Upon expansion, the radially expandable implant extends through the sinus tarsi, contacting both the calcaneus and talus, thus altering the range of motion of the subtalar joint. The expanded implant thus alters the alignment of the foot and provides resistance against foot pronation.

After the implant has been inserted, the skin wound made by the delivery tool is closed and allowed to heal over the sinus tarsi. With the employment of the minimally invasive percutaneous procedure, which excludes all post-implantation communication with a contaminated skin surface, the present invention provides rapid arthroereisis of the subtalar joint, and allows mobilization of the patient's limb in minimal time and with a lower infection risk. Thus, when the implant is used to treat flat feet, the patient can begin to move the extremity very shortly after the insertion. Such rapid mobilization promotes healing and reduces muscle atrophy. The patient regains use of the treated foot as quickly as possible. Even more importantly, healing proceeds without the need for extensive physiotherapy, which is typically required after the prolonged periods of immobilization commonly encountered when patients are treated with existing subtalar joint implants.

In the preferred embodiments, the implant is made of bio-compatible metals like Nitinol, titanium, S.S. 316 or suitable polymers. Preferably, after insertion, the radial expansion of the implant is such that its diameter substantially increases. Thus, the diameter can increase by at least 50%, by 100%, by 200%, or more if desired. This large factor of expansion is advantageous in that during insertion, the unexpanded implant is narrow enough to fit easily through a small skin incision. In contrast, the implant expands after placement such that its diameter fills substantially all of the sinus tarsi so that the subtalar joint motion and alignment is altered.

Thus, more generally, the initial size of the implant maintains a reduced diameter small enough to be passed through a needle so as to be inserted into a bone through a syringe or other delivery tool, and is capable of expanding to an expanded diameter large enough to fill substantially the sinus tarsi of the foot. The implant is preferably substantially frusta-conical in shape after expansion, but other geometric shapes are also provided, including but not limited to cubes, cylinders, and others.

In some preferred embodiments of the present invention, the subtalar implant comprises a self-expanding structure. In the context of the patent application and the claims, the term “self-expanding” or “self-expandable” is used to mean that once the implant is inserted into the desired location, it expands radially outward due to mechanical force generated by the implant itself. This mechanical force may be due to potential energy stored in the implant, for example, as a result of radially compressing the implant before inserting it into the cavity. Additionally or alternatively, as described below, the implant may expand due to heat absorbed by the implant in the sinus tarsi. As disclosed below, certain preferred configurations and materials are used to provide this self-expanding effect. Subtalar implants in accordance with these preferred embodiments differ from expandable subtalar implants known in the art, which require external application of mechanical force to the implant to cause the implant to expand within the sinus tarsi.

Before introduction into the foot, the self-expanding implant is preferably compressed radially inward into a closed, reduced cross sectional configuration and is inserted or attached to the catheter in this closed, reduced cross sectional configuration. The implant then expands radially outward, to bear against and realign the foot. After the implant is put into place, the catheter is withdrawn, leaving the implant behind in the foot. Thus, the structure and the material from which it is produced, as described below, should generally be sufficiently flexible to be compressed into the closed, reduced configuration, but rigid enough to alter the foot alignment in the open, expanded configuration.

In some preferred embodiments of the self-expanding implants, the implant comprises a resilient or elastic, biocompatible material. Preferably, the resilient or elastic material is a superelastic or shape memory material, for example, Nitinol, or another metal, such as titanium, or else a polymer material. The implant is fabricated, as is known in the art, so as to exert an outward radial force when compressed.

In other embodiments, the implant comprises a biocompatible shape memory material, likewise such as Nitinol. Preferably, the material is chosen and prepared, as is known in the art, so that upon compression of the implant into its closed, reduced configuration, the material assumes a state of stress-induced martensite, wherein it is relatively flexible and elastic. When released inside the sinus tarsi, the implant springs back to its desired shape, the open, expanded configuration, and the material assumes an austenitic state, wherein it is substantially rigid and alters subtalar alignment and foot motion.

The structure of the implant itself can be formed by tightly rolling together one or more sheets or ribbons of self-expanding material, preferably superelastic or shape memory material, as described above, to form a generally conical spiral structure. After insertion of the implant into the sinus tarsi, the spiral partially unrolls as it expands radially outward, until it has expanded to substantially fill the sinus tarsi. Preferably, at least one edge of each of the one or more sheets of the material is bent so as to protrude radially outward from the outer, radial surface of the spiral. As the spiral expands, these protruding edges engage the inner surface of the talus and calcaneus, so as to anchor the implant firmly in place and prevent sliding or rotation of the implant out of the sinus tarsi. More preferably, two or more of the edges are bent at different angles, in order to prevent rotation of the bone in either a clockwise or a counterclockwise direction.

In other preferred embodiments of the invention, the implant includes a holding device, for example, a pin, which is fitted into the implant before insertion of the implant into the foot. The holding device is fitted into the implant while the implant is held mechanically in its compressed, closed configuration and then continues to hold the implant in this configuration. After the implant has been inserted and properly placed in the sinus tarsi, the holding device is withdrawn, and the implant self-expands radially outward to anchor itself in place and fixate the bone. In an alternate embodiment, the holding device comprises an outer sheath of the delivery tool to resist radial expansion of implant until the outer sheath is withdrawn.

As an alternative to a self-expanding implant, the implant can be constructed to be expandable by the application of energy or external power. For example, the shape memory material can be chosen and prepared, as is known in the art, so as to have a critical temperature of approximately 30 degrees Celsius. Thus, at room temperature, the material is normally at least partially in a martensitic state, so that the implant remains flexible and elastic before its insertion into the bone. When inserted into the bone, the implant becomes exposed to body temperature, at which temperature, the material assumes at least a partially austenitic state, and the implant is substantially rigid.

In such embodiments, wherein heat is applied to the implant to cause it to expand, instead of, or in addition to the use of body temperature, after the implant is inserted into the sinus tarsi and the catheter is withdrawn, an external heat source can be used for the application of heat. This can be accomplished, for example, through a heating probe that is brought into contact with the implant. The heat causes the implant to expand radially outward and to become substantially rigid, so as to anchor itself in place and alter subtalar motion. The heating probe or other heat source is then removed.

In other preferred embodiments of the present, the implant comprises a conical tube, made of stiff, resilient material, as described above, and having a plurality of openings through its radial wall, so that the wall has substantially the form of a meshwork. The meshwork preferably comprises a plurality of longitudinal ribs, interconnected by generally arcuate circumferential struts. When the implant is radially compressed, the struts are bent inward, toward the central axis of the tube. The holding device, preferably a pin, is inserted along the axis and holds the struts in their bent configuration, thus preventing the implant from expanding. When the pin is removed, with the implant inside the bone, the struts resume substantially their arcuate shape, with the implant either self-expanding radially outward, or expanding due to the application of energy, until the implant engages the inner bone surface adjoining the sinus tarsi.

Over time, after insertion of the implant in the sinus tarsi, the surrounding tissue will tend to grow into and through the openings in the mesh-like wall of the implant, so that the overall structure of the implant will be strengthened.

In another embodiment of the invention, the implant comprises a plurality of leaves, which are bent so that the inner end of each leaf normally extends radially outward, away from a central, longitudinal axis of the implant. The leaves are arranged along the axis in a generally spiral pattern, wherein each leaf extends outward at a different angle relative to a reference point on the axis from one or more other leaves that axially adjoin it. Preferably, the outer end of each leaf curves radially inward. Before inserting the implant into the foot, the implant is compressed by bending the leaves inward, to form a narrow, generally tubular shape. The holding device, preferably a pin, in then inserted along the axis of the tubular shape, so as to engage and hold the inward curved outer ends of the leaves and prevent their radial expansion. After the implant has been inserted into the sinus tarsi, the pin is withdrawn, and the leaves snap back radially outward, engaging the inner bone surface and anchoring the implant in place.

Alternatively, in other embodiments of the invention involving the application of external energy, a balloon may be inserted inside the implant and inflated to expand the implant. After the implant is expanded, the balloon is preferably deflated and withdrawn although it can also be left implanted. In other embodiments, the balloon may be left in place and detached from the catheter to further support the implant.

In one embodiment, a method for treating a patient is provided, comprising the steps of providing a self-expandable subtalar implant, inserting said implant into the sinus tarsi of a foot, and allowing self-expansion of said implant in the sinus tarsi. The method may further comprise changing the alignment of the hindfoot. The inserting step may performed through a cannula inserted into said sinus tarsi of said patient, or over a guidewire inserted into said sinus tarsi of said patient. The method may further comprise inserting a balloon catheter in said implant, and expanding the balloon of said catheter. The method may further comprise detaching said balloon from said catheter.

In one embodiment, a method for treating a patient is provided, comprising providing a self-expanding subtalar implant, identifying a foot having a first range of motion, inserting said implant into the sinus tarsi of said foot, and adapting said foot to a second range of motion by allowing self-expansion of said implant.

In another embodiment, a method for treating a patient is provided, comprising providing a self-expandable subtalar implant, identifying a foot having a first weight-bearing alignment, limiting said foot to a second weight-bearing alignment, inserting said implant into a sinus tarsi of a foot, and securing said foot in said second weight-bearing alignment by allowing self-expansion of said implant. The first and second weight-bearing alignments may be defined by the angle between a first line connecting the edges of an articular surface of a talus and a second line connecting the edges of an articular surface of a navicular bone.

In one embodiment, a method for treating a patient is provided, comprising the steps of providing an expandable subtalar implant with an internal lumen, inserting said implant into the sinus tarsi of a foot, and expanding said implant by plastic deformation of at least a portion of said implant. The method may further comprise changing the alignment of the hindfoot. The inserting step may be performed through a cannula inserted into said sinus tarsi of said patient or over a guidewire inserted into said sinus tarsi of said patient. The expanding step may performed by a balloon catheter.

In another embodiment, a method for treating a patient is provided, comprising providing an expandable subtalar implant, identifying a foot having a first range of motion, inserting said implant into the sinus tarsi of said foot, and adapting said foot to a second range of motion by deformably expanding said implant. The expandable subtalar implant of the providing step may have a first end, a second end and a middle deformable portion that is capable of radial expansion by moving the first end and second end in closer proximity. The expanding step may comprise moving the first end and the second end of said implant in close proximity.

In another embodiment, a method for treating a patient is provided, comprising the steps of providing an expandable subtalar implant, identifying a foot having a first weight-bearing alignment, limiting said foot to a second weight-bearing alignment, inserting said implant into a sinus tarsi of a foot, and securing said foot in said second weight-bearing alignment by deforming expansion of said implant. The first and second weight-bearing alignments may be defined by the angle between a first line connecting the edges of an articular surface of a talus and a second line connecting the edges of an articular surface of a navicular bone, by the angle between a first line along the long axis of a talus and a second line along the long axis of a first metatarsal bone, by the angle between a first line between the plantar-most point of a calcaneus of a patient and an most inferior point of the distal articular surface of said calcaneus, and a second line within a horizontal plane of said patient, or by the angle between a first line along the plantar border of a calcaneus and a second line along a first midpoint in the body of a talus and a second midpoint in the neck of said talus.

In one embodiment, a method for treating a patient is provided, comprising the steps of identifying a cyma line in a foot of a patient, smoothing said cyma line, and securing said smoothing by expanding an implant in the sinus tarsi of said foot.

In another embodiment, a method of treating a patient is provided, comprising the steps of accessing a sinus tarsi of a foot through an access path having a cross sectional diameter of no more than about 0.5 inches, the sinus tarsi having a talus and calcaneus spaced apart by a first minimum distance, increasing the space between the talus and calcaneus to a second minimum distance, and restraining the talus and calcaneus at said second minimum distance.

In one embodiment, a method for treating a patient is provided, comprising providing an expandable first metatarsal-phalangeal joint implant, inserting said implant into a first metatarsal-phalangeal joint of a foot, and expanding said implant with a fluid.

In another embodiment, a method for treating a patient is provided, comprising providing a mass-increasable subtalar implant, inserting said implant into the sinus tarsi of a foot, and allowing self-expansion of said implant in the sinus tarsi. The method may further comprise changing the alignment of the hindfoot. In one embodiment, the inserting step may be performed through a cannula inserted into said sinus tarsi of said patient, or over a guidewire inserted into said sinus tarsi of said patient. In a further embodiment, the method may further comprise inserting a balloon catheter in said implant, and expanding the balloon of said catheter. In still a further embodiment, the method may further comprise detaching said balloon from said catheter.

In one embodiment, a method for treating a patient is provided, comprising the steps of providing a mass-increasable subtalar implant, identifying a foot having a first range of motion, inserting said implant into the sinus tarsi of said foot, and adapting said foot to a second range of motion by increasing the mass of said implant.

In one embodiment, a method for treating a patient is also provided, comprising providing a mass-increasable subtalar implant, identifying a foot having a first weight-bearing alignment, limiting said foot to a second weight-bearing alignment, inserting said implant into a sinus tarsi of a foot, and securing said foot in said second weight-bearing alignment by increasing the mass of said implant. The first and second weight-bearing alignments may be defined by the angle between a first line connecting the edges of an articular surface of a talus and a second line connecting the edges of an articular surface of a navicular bone.

In one embodiment, a method for treating a patient is provided, comprising providing an inflatable subtalar implant, inserting said implant into the sinus tarsi of a foot, and inflating said implant with an inflation material. The inflation material may be a fluid or a solid. The solid may comprise microspheres. The method may further comprise changing the alignment of the hindfoot. The inserting step may be performed through a cannula inserted into said sinus tarsi of said patient. The inserting step may be performed over a guidewire inserted into said sinus tarsi of said patient. The method may further comprise combining multiple agents to form said inflation material. The combining step may be performed before said inflating step or during said inflating step.

In another embodiment, a method for treating a patient is provided, comprising the steps of providing an inflatable subtalar implant, identifying a foot having a first range of motion, inserting said implant into the sinus tarsi of said foot, and adapting said foot to a second range of motion by inflating said implant.

In another embodiment, a method for treating a patient is provided, comprising providing an inflatable subtalar implant, identifying a foot having a first weight-bearing alignment, limiting said foot to a second weight-bearing alignment, inserting said implant into a sinus tarsi of a foot, and securing said foot in said second weight-bearing alignment by inflating said implant. The first and second weight-bearing alignments may be defined by the angle between a first line connecting the edges of an articular surface of a talus and a second line connecting the edges of an articular surface of a navicular bone, by the angle between a first line along the long axis of a talus and a second line along the long axis of a first metatarsal bone, by the angle between a first line between the plantar-most point of a calcaneus of a patient and a most plantar point of the distal articular surface of said calcaneus, and a second line within a horizontal plane of said patient, or by the angle between a first line along the plantar border of a calcaneus and a second line along a first midpoint in the body of a talus and a second midpoint in the neck of said talus.

In one embodiment, a minimally invasive method for treating a patient is provided, comprising the steps of providing an inflatable subtalar implant, inserting said implant into a sinus tarsi of a foot, inflating said implant, changing the range of motion of the subtalar joint of said foot, and conforming the implant to the shape of the sinus tarsi thereby.

In one embodiment, a method for treating a patient is provided, comprising the steps of identifying a cyma line in a foot of a patient, smoothing said cyma line, and securing said smoothing by inflating an implant in the sinus tarsi of said foot.

In another embodiment, a method of treating a patient is provided, comprising the steps of accessing a sinus tarsi of a foot through an access path having a cross sectional diameter of no more than about 0.5 inches, the sinus tarsi having a talus and calcaneus spaced apart by a first minimum distance, increasing the space between the talus and calcaneus to a second minimum distance, and restraining the talus and calcaneus at said second minimum distance.

In another embodiment, a method for treating a patient is provided, comprising the steps of providing an inflatable first metatarsal-phalangeal joint implant, inserting said implant into a first metatarsal-phalangeal joint of a foot, and inflating said implant with a fluid.

In one embodiment of the invention, a subtalar joint implant is provided, comprising an inflatable balloon adapted for positioning in the sinus tarsi of a foot.

In another embodiment, a foot implant is provided, comprising an inflatable balloon, wherein said inflatable balloon is adapted for extra-articular positioning in the sinus tarsi of the foot.

Several embodiments of the invention provide these advantages, along with others that will be further understood and appreciated by reference to the written disclosure, figures, and claims included herein.

### BRIEF DESCRIPTION OF THE DRAWINGS

The structure and method of making the invention will be better understood with the following detailed description of embodiments of the invention, along with the accompanying illustrations, in which:

FIG. 1 is a superior elevation view of the calcaneus;

FIG. 2 is a lateral elevation view of the talo-calcaneus relationship;

FIG. 3 is a lateral elevation view of the foot bones showing the sinus tarsi;

FIG. 4 is dorso-plantar elevation view of the foot showing the outline of the sinus tarsi;

FIG. 5A is a superior elevation view of the ligament attachment sites to the calcaneus; FIG. 5B is a coronal cross-section view showing the ligaments of the sinus tarsi;

FIGS. 6A and 6B depict the axis of rotation for the subtalar joint;

FIGS. 7A and 7B are schematic views of the motion of the subtalar joint as a mitered hinge joint;

FIGS. 8A and 8B are schematic views of subtalar joint motion as a threaded screw joint;

FIGS. 9A and 9B are posterior cross-sectional views of a neutrally aligned and a hyperpronated foot;

FIGS. 10A and 10B are lateral radiographs of the foot illustrating the cyma lines in a neutrally aligned and misaligned foot, respectively;

FIGS. 11A and 11B are AP radiographs of the foot illustrating the cyma lines in a neutrally aligned and misaligned foot, respectively;

FIGS. 12A and 12B are AP radiographs of the foot depicting the talonavicular coverage angles in a neutrally aligned and misaligned foot, respectively;

FIGS. 13A and 13B are lateral radiographs of the foot depicting lateral talocalcaneal angles in a neutrally aligned and misaligned foot, respectively;

FIGS. 14A and 14B are lateral radiographs of the foot depicting the calcaneal pitch angles in a neutrally aligned and misaligned foot, respectively;

FIGS. 15A and 15B are AP radiographs of the foot depicting AP-talar-first metatarsal angles in a neutrally aligned and misaligned foot, respectively;

FIGS. 16A and 16B are lateral radiographs of the foot depicting the lateral talocalcaneal angles in a neutrally aligned and misaligned foot, respectively;

FIGS. 17A and 17B are AP radiographs of the foot depicting AP talocalcaneal angles in a neutrally aligned and misaligned foot, respectively;

FIGS. 18A and 18B are schematic coronal cross-sectional views of a neutrally aligned and hyperpronated foot, respectively. FIG. 18C is a schematic view depicting the effect of material placed within the sinus tarsi. FIG. 18D is a schematic view depicting the tendency of the talus and calcaneus to cause displacement of material in the sinus tarsi;

FIGS. 19A and 19B are schematic longitudinal cross-sectional views of the talus and calcaneus in a hyperpronated foot before and after insertion of material into the sinus tarsi;

FIGS. 20A and 20B are side elevation and cross-sectional views of one embodiment of the implant;

FIGS. 21A through 21H depict side elevation views of various embodiments of non-conforming implants;

FIGS. 22A and 22B are elevation and cross sectional views of one embodiment of the invention having a ridged outer surface;

FIGS. 23A and 23B are cross-sectional views of the foot with various embodiments of barbs for anchoring the implant;

FIGS. 24A and 24B represent various embodiments of the invention comprising multiple inflatable compartments;

FIGS. 25A and 25B are elevation views of one embodiment of the coupling interface and the distal end of a complementary delivery catheter. FIG. 25C is a cross-sectional view of the implant in FIGS. 25A and 25B attached to a delivery catheter;

FIGS. 26A and 26B are elevation views of another embodiment of the coupling interface and the distal end of a complementary delivery catheter. FIG. 26C is a cross-sectional view of the implant in FIGS. 26A and 26B attached to a delivery catheter;

FIGS. 27A through 27C depict one embodiment of the delivery system;

FIGS. 28A and 28B are schematic cross-sectional views of the foot before and after inflation of the sizing catheter;

FIG. 29 is a side elevation view of a foot following insertion of the delivery catheter;

FIGS. 30A and 30B are schematic cross-sectional views of the foot with the implant inserted; FIG. 30A shows an uninflated implant attached to the delivery catheter and FIG. 30B depicts an inflated implant with the delivery catheter removed;

FIG. 31A is a front elevation view of one embodiment of a first MTP joint inflatable implant and FIG. 31B is a side cross-sectional view of the implant in FIG. 31A;

FIG. 32 is a schematic, isometric view of one embodiment of a self-expanding subtalar implant, in accordance with a preferred embodiment of the present invention;

FIG. 33A is a schematic, sectional illustration of one embodiment showing a self-expanding subtalar implant in a first, closed configuration; FIG. 33B is a schematic, sectional illustration showing the implant of FIG. 33A in a second, open configuration;

FIGS. 34A through 34C are schematic, sectional illustrations showing the use of the implant of FIG. 32 in the sinus tarsi;

FIG. 35A is a schematic, isometric representation of another embodiment of a self-expanding subtalar implant, in an open configuration; FIG. 35B is a schematic, sectional illustration showing the implant of FIG. 35A in a closed configuration, wherein a holding pin is inserted along a central axis of the implant;

FIG. 36A is a schematic, end view of another embodiment, comprising a self-expanding subtalar implant, in an open configuration; FIG. 36B is a schematic illustration showing preparation of material for fabrication of the implant shown in FIG. 36A; FIG. 36C is a schematic, sectional view of the implant of FIG. 36A, in a closed configuration with an internal holding pin;

FIGS. 37A through 37D are perspective views of two subtalar implants in open and closed positions; These devices can be opened by a transfer of heat (e.g. if they are constructed from shape memory material), can be opened by use of a balloon, or by any additional suitable mechanical method. FIGS. 37A and 37B are illustrations of one embodiment of the device, shown in compressed and expanded configurations respectively; FIGS. 37C and 37D are illustrations of another embodiment of the device, shown in compressed and expanded configurations, respectively;

FIGS. 38A and 38B are schematic cross sectional illustrations of one embodiment of the invention comprising a subtalar joint implant whose height can be mechanically varied; It is shown in closed (FIG. 38A) and open (FIG. 38B) configurations. The device can include hinges at its joints or joints that undergo plastic deformation;

FIG. 39 is a schematic isometric view of one embodiment of the invention;

FIG. 40A shows a schematic cross sectional view of another embodiment of the invention; FIG. 40B is a schematic cross sectional view of the implant of FIG. 40A; FIG. 40C is a sectional view of a modified version of the implant of FIGS. 40A and 40B, shown in its expanded state, with multiple locking mechanisms;

FIG. 41A shows two cross sectional views of another embodiment of the subtalar implant; Cross sectional views of both the constricted and expanded configurations are shown, with these constricted and expanded configurations being superimposed for comparison purposes; FIG. 41B presents two cross sectional views of another embodiment of the subtalar implant; As in FIG. 41A, cross sectional views of both the constricted and expanded configurations are shown superimposed for comparison purposes;

FIGS. 42A and 42B illustrate one embodiment of the invention with a medial longitudinal canal. The canal allows insertion of the implant on a guidewire to facilitate positioning; FIG. 42A is a perspective view of the implant, having the canal therein, and FIG. 42B is a schematic of the implant, showing the canal extending therethrough;

### DETAILED DESCRIPTION OF THE PREFERRED EMBODIMENT

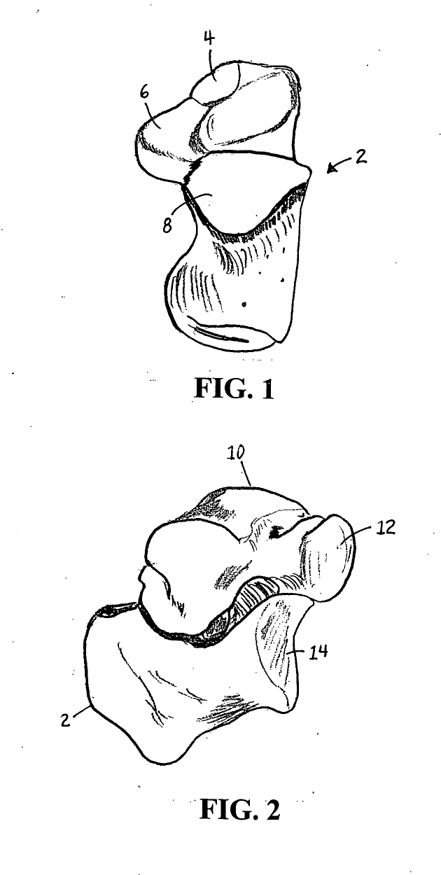

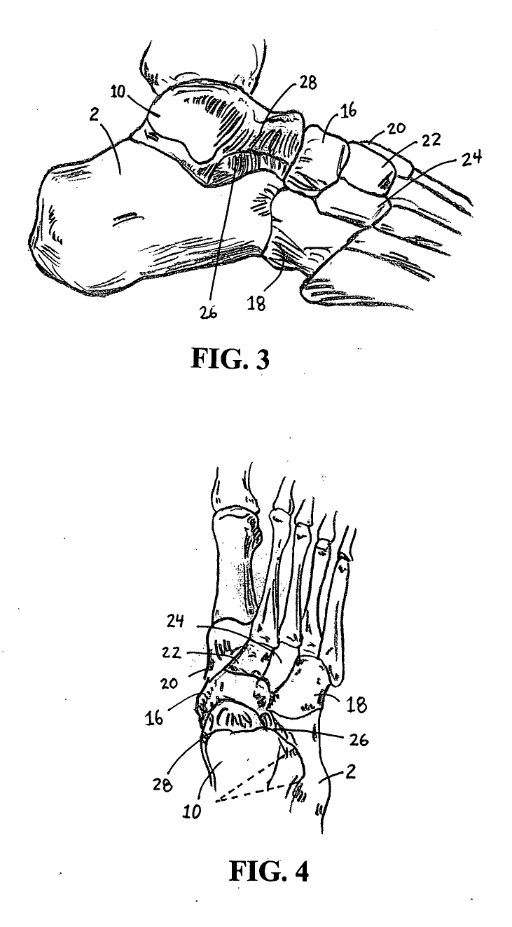

The talus and calcaneus form the bones of the hindfoot. The talus is a bone with no muscular attachments, but is stabilized by ligaments and cradled by the tendons passing from the leg to the foot. As shown in FIG. 1, the calcaneus 2 articulates with the talus at the calcaneal anterior 4, middle 6 and posterior facets 8. FIG. 2 depicts the relationship between the talus 10 and calcaneus 2 and the talo-calcaneal surfaces 12, 14 that articulate with the midfoot bones. FIGS. 3 and 4 depict the midfoot bones, including the navicular 16, cuboid 18 and cuneiform bones 20, 22, 24. The sinus tarsi 26, also known as the talocalcaneal sulcus, is an extra-articular anatomic space between the inferior neck 28 of the talus 10 and the superior aspect of the distal calcaneus 2. The space continues with the tarsal canal, a funnel or trumpet-shaped space that extends medially to a small opening posterior to the sustentaculum tali. Sinus tarsi 26 is oriented obliquely from a lateral distal opening to proximal medial end. The canal is wider laterally and narrower medially, but the lateral opening of the canal is capable of widening with foot supination and narrowing with pronation. Fat and ligaments occupy the space and are perfused by the tarsal canal artery, a branch of the posterior tibial artery.

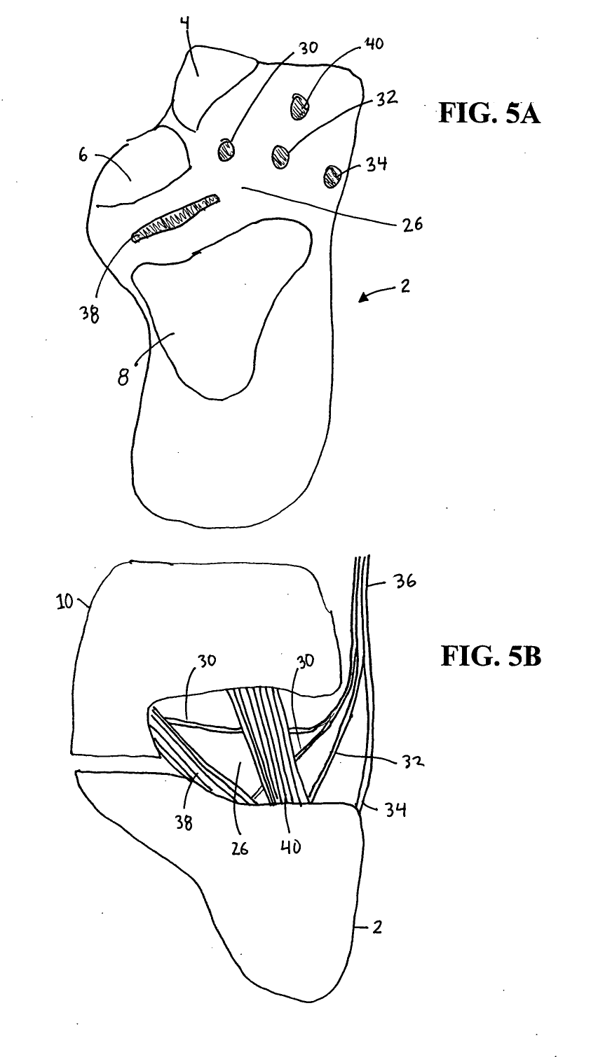

FIG. 5A is a superior view of the calcaneus 2 showing the ligament attachments within the tarsal canal, including the inferior attachments 30, 32, 34 of the extensor retinaculum 36 of the foot, the interosseous talocalcaneal ligament 38 and the cervical ligament 40. The primary ligament is interosseous talocalcaneal ligament 38, shown in a coronal cross section of the foot in FIG. 5B. Its primary function is to maintain apposition of the talus 10 to the calcaneus 2. The interosseous talocalcaneal ligament 38 is anterior to the posterior subtalar joint and extends from calcaneus 2 to talus 10. It forms a transverse partition between the sulcus tali and the sulcus calcaneus, the two grooves forming the sinus tarsi. Interosseus ligament 38 separates anterior 4 and middle facets 6 of the calcaneal portion of the anterior subtalar joint from the posterior facet 8 of the posterior subtalar joint and provides stability to the hindfoot. The cervical ligament 40, like the other ligaments of the tarsal sinus 26, is extra-capsular. Cervical ligament 40 is larger than interosseous talocalcaneal ligament 38. It attaches to the cervical tubercle of the inferior and lateral aspects of neck 28 of talus 10 and the dorsal aspect of calcaneus 2 medial to the origin of the extensor digitorum brevis muscle. Cervical ligament 40 is flattened, its width being four times greater than its thickness. The primary function of cervical ligament 40, along with interosseous talocalcaneal ligament 38, is to limit inversion of the hindfoot. The inferior extensor retinaculum 36 is a Y-shaped strap of flat thick connective tissue that crosses the proximal portion of the foot. The stem of the “Y” is composed of superficial and deep laminae that enclose the long extensor tendons and prevent bow stringing of the long extensor tendons. Laterally, inferior extensor retinaculum 36 is anchored to talus 10 and calcaneus 2 by ligament-like roots that are located in the tarsal sinus and canal. The medial 30, intermediate 32 and lateral roots 34 together constitute the majority of the ligamentous material in the tarsal sinus 26. Inferior extensor retinaculum 36 assists cervical ligament 40 in limiting inversion of the subtalar joint. Medial root 30 attaches to calcaneus 2 just anterior to the attachment site of interosseous talocalcaneal ligament 38. Medial root 30 has a secondary attachment site to talus 10 in common with interosseous talocalcaneal ligament 38. Intermediate root 32 attaches to calcaneus 2 posterior to the attachment site of cervical ligament 40. Lateral root 34 attaches to calcaneus 2 at the external aspect of the tarsal sinus 26.

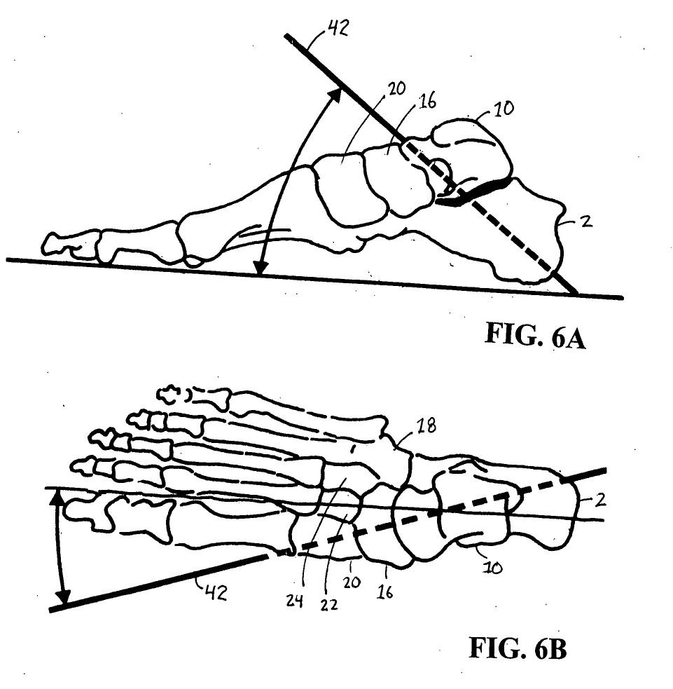

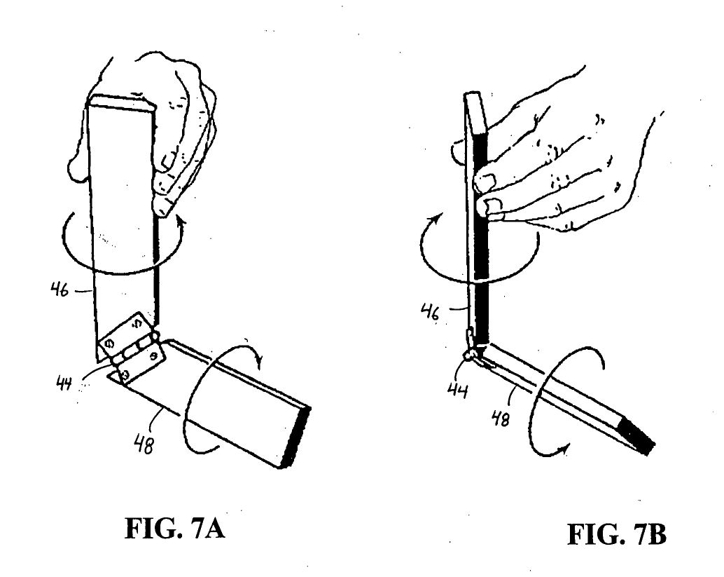

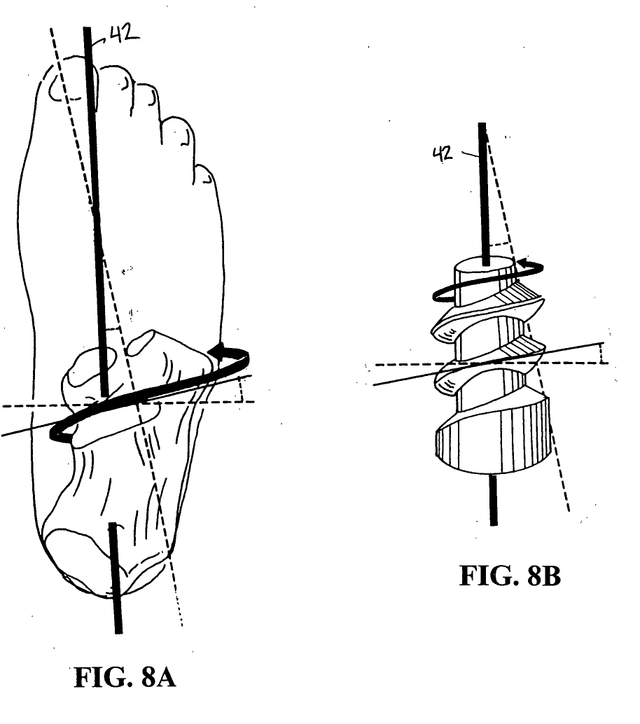

Subtalar motion is generally described as a rotational motion of the talus around the calcaneus. FIGS. 6A and 6B depict the subtalar axis of rotation 42, which typically extends upward and forward at an angle of about forty-two degrees from the floor at the heel. The axis deviates sixteen degrees medially from the midline of the foot. Generally, the subtalar joint can be inverted about twenty degrees and everted about five to ten degrees. The average range of motion throughout the stance phase of gait, however, is only about six degrees. Longitudinal translation in both the proximal and distal directions is also associated with the rotation movement, but the direction and magnitude of this movement is highly variable in each person. Some researchers have characterized the motion of the subtalar joint as a mitered hinge joint 44, as shown in FIGS. 7A and 7B. The vertical member 46 is analogous to the leg and the horizontal member 48 is analogous to the foot. Other researchers, however, have characterized the motion of the subtalar joint as a screw joint, as shown in FIGS. 8A and 8B. The differences between the characterizations of the subtalar joint underscore the high degree of variation in the configuration of the joint within the population.

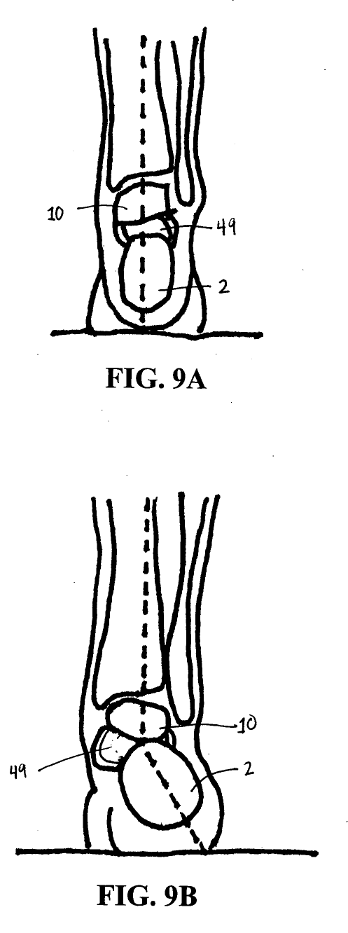

When an excessive range of motion exists in the subtalar joint, misalignment of the foot can occur. Compared to a person with a neutrally aligned foot, shown in FIG. 9A, a person with flat feet, shown in FIG. 9B, has a subtalar joint that is capable of eversion up to about six degrees or more from a neutral talo-calcaneal alignment. Excessive eversion places increased stress upon the foot arch. Over time, foot or ankle disorders can develop from the misalignment. Misalignment of the subtalar joint also affects the alignment of the bones in the midfoot due to the dependence of midfoot stability on hindfoot stability.

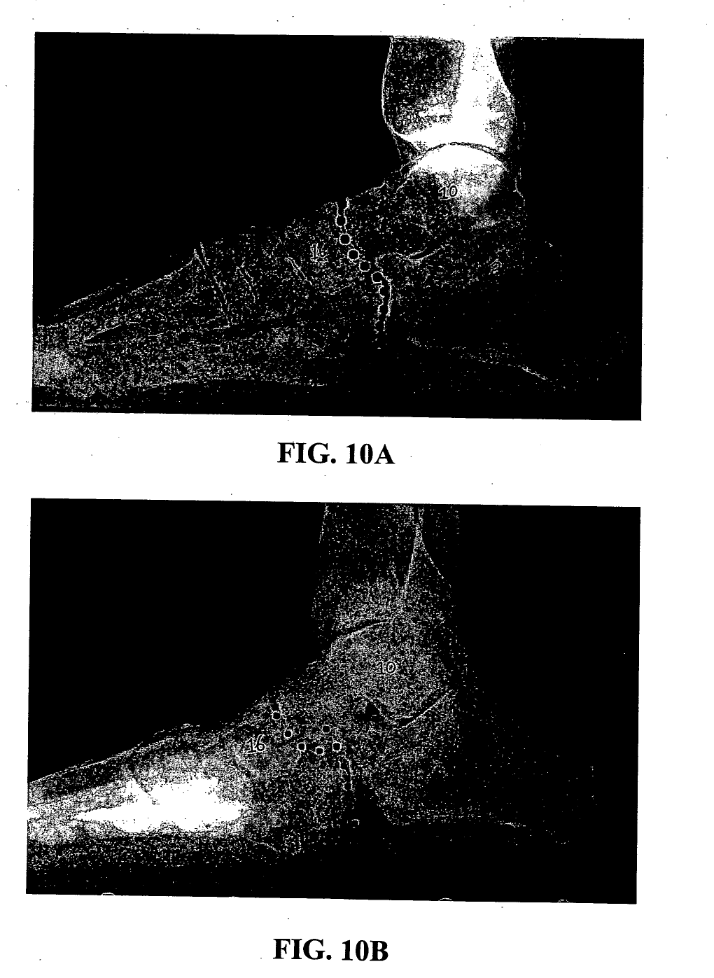

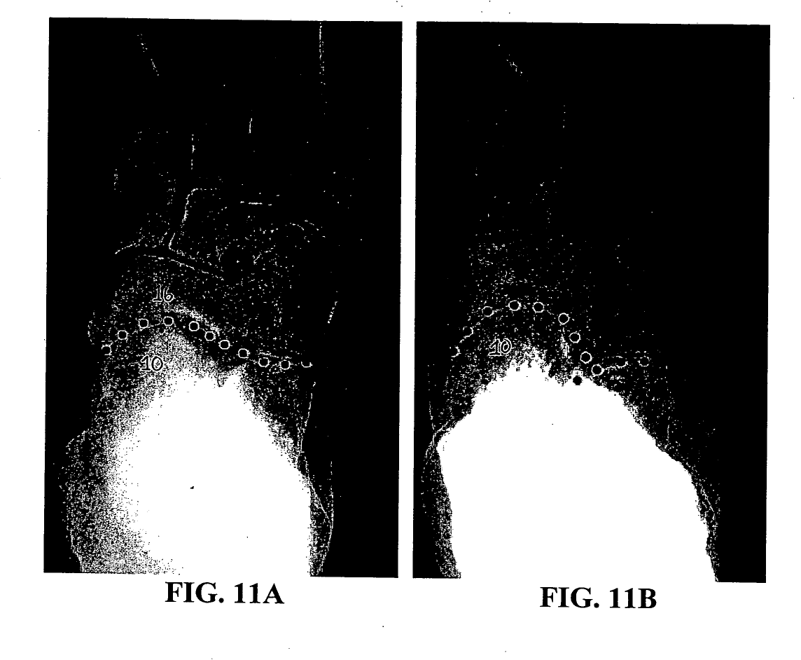

Alignment of the foot can be assessed on plain film x-ray imaging by examining the cyma lines of the foot. The term “cyma line” refers to the joining of two curved lines. A neutrally aligned foot forms a smooth cyma line (shown with dots) between the talonavicular joint and the calcaneocuboid joint on radiographs in both the lateral and AP views, as shown in FIGS. 10A and 11A, respectively. If the cyma line is broken, as shown in FIGS. 10B and 11B, this finding suggests misalignment of the talus 10 on the calcaneus 2 as seen in patients with flat feet.

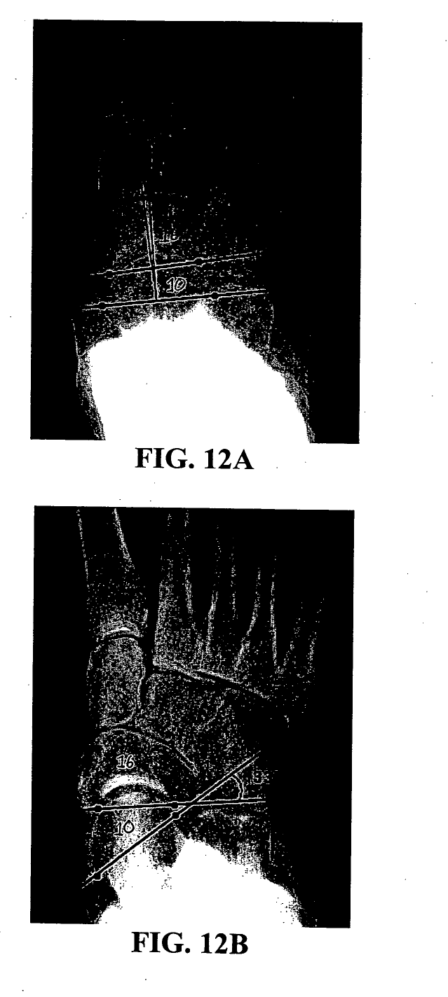

Other radiographic methods of assessing foot alignment are also available. FIGS. 12A and 12B depict the evaluation of talonavicular uncoverage. Talonavicular uncoverage is an indication of forefoot abduction, a component of flatfoot. This measurement is taken from a weight-bearing AP view. This angle represents the degree of shift of navicular 16 on talus 10. Two lines are drawn, one connecting the edges of the articular surface 52 of the talus 10, and one connecting the edges of the articular surface 54 of the navicular 16. The angle formed by these two lines is the talonavicular coverage angle, as seen in FIG. 12A. An angle of at least about 7 degrees indicates lateral talar subluxation, shown in FIG. 12B. In one embodiment of the invention, a subtalar implant is configured in the sinus tarsi to correct the talonavicular coverage angle to about 15 degrees or less. In another embodiment, the implant is configured in the sinus tarsi to correct the talonavicular coverage angle to about 8 degrees or less. In still another embodiment, the implant is configured in the sinus tarsi to correct the talonavicular coverage angle to about 5 degrees or less.

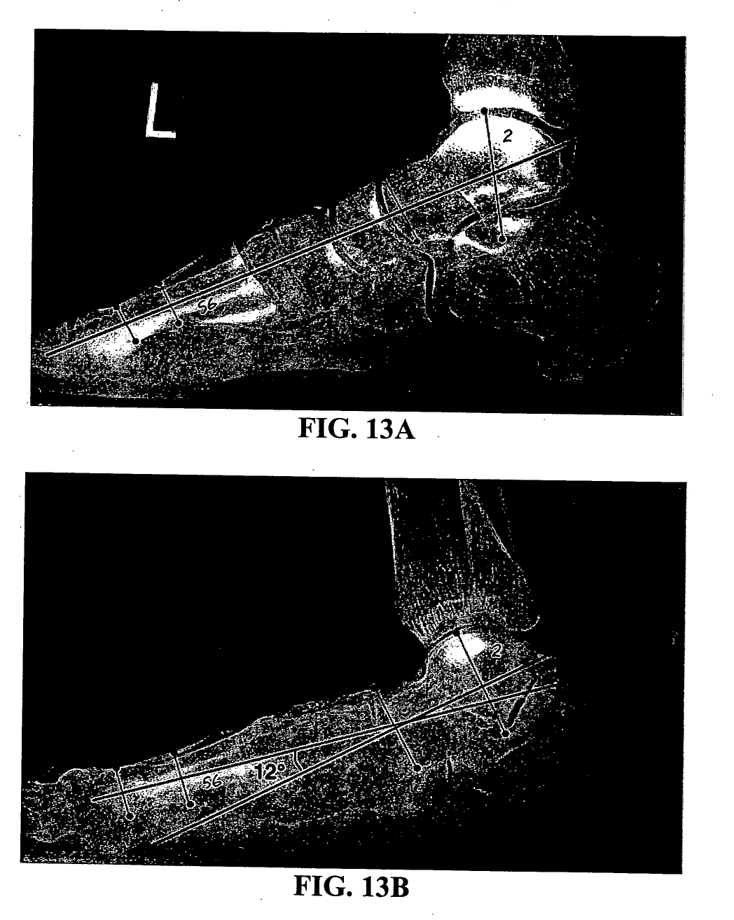

A more direct measurement of pes planus, or collapse of the longitudinal arch, is the talar-first metatarsal angle (Meary's angle), shown in FIGS. 13A and 13B. This is an angle formed between the long axis of the talus 2 and first metatarsal 56 on a weight-bearing lateral view. This line is used as a measurement of collapse of the longitudinal arch 50. Collapse may occur at the talonavicular joint, naviculo-cuneiform, or cuneiform-metatarsal joints. In the normal weight-bearing foot, shown in FIG. 13A, the midline axis of the talus 2 is in line with the midline axis of the first metatarsal 56. A drop in angle of at least about 4° convex downward is considered pes planus. An angle of at least about fifteen to about thirty degrees, as in FIG. 13B, is considered moderate flat foot, and an angle of at least about 30° is considered severe flat foot. In one embodiment of the invention, a subtalar implant is configured in the sinus tarsi to correct Meary's angle to about a downward 50 degrees or less. In another embodiment, the implant is configured in the sinus tarsi to correct Meary's angle to about a downward 25 degrees or less. In still another embodiment, the implant is configured in the sinus tarsi to correct Meary's angle to about a downward 5 degrees or less. In still another embodiment, the implant is configured in the sinus tarsi to correct Meary's angle to about zero degrees. In still another embodiment, the implant is configured in the sinus tarsi to correct Meary's angle to about an upward 5 degrees or more.

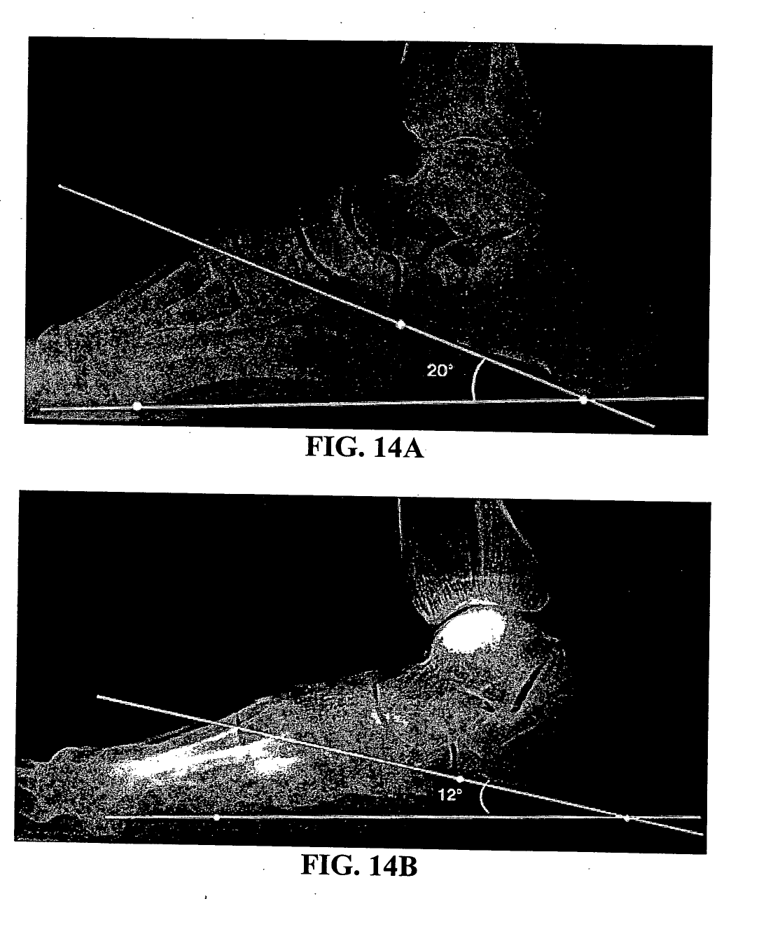

FIGS. 14A and 14B depict radiographs evaluating the calcaneal inclination angle, or calcaneal pitch. A line is drawn from the plantar-most surface of the calcaneus 2 to the inferior border of the distal articular surface. The angle created between this line and the transverse plane, or the line from the plantar surface of the calcaneus 2 to the inferior surface of the fifth metatarsal head, is the calcaneal pitch, shown in FIG. 14A. A decreased calcaneal pitch is consistent with pes planus, as represented in FIG. 14B. There have been differing opinions between researchers concerning the normal range of calcaneal pitch. About eighteen to about twenty degrees is generally considered normal, although measurements ranging from about seventeen to about thirty-two degrees have also been reported to be normal. In one embodiment of the invention, a subtalar implant is configured in the sinus tarsi to correct calcaneal pitch to about 10 degrees or more. In another embodiment, the implant is configured in the sinus tarsi to correct calcaneal pitch to about 15 degrees or more. In still another embodiment, the implant is configured in the sinus tarsi to correct calcaneal pitch to about 20 degrees or more.

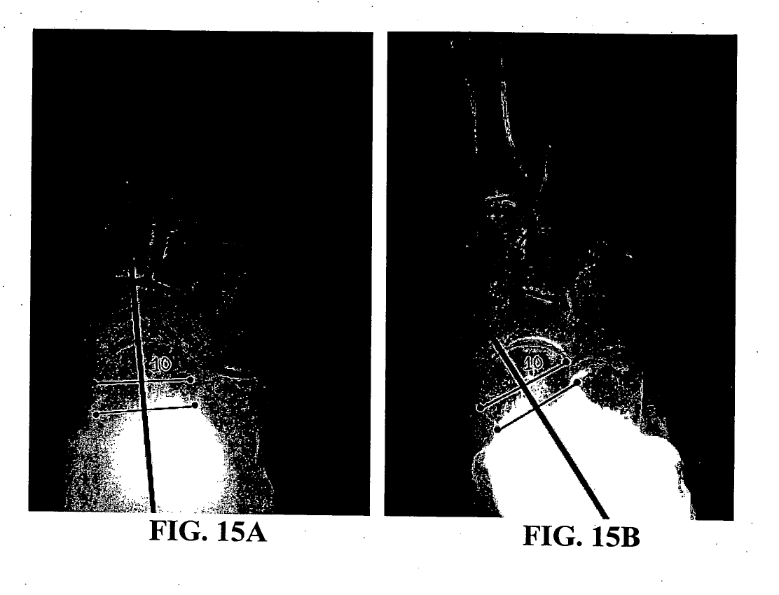

FIGS. 15A and 15B depict radiographs evaluating the AP-talar-first metatarsal angle. A line drawn through the mid-axis of the talus 10 should be in line with the first metatarsal shaft 56, as in FIG. 15A. If the line is angled medial to the first metatarsal 56 it indicates pes planus, as illustrated in FIG. 15B. In one embodiment of the invention, a subtalar implant is configured in the sinus tarsi to correct the AP-talar-first metatarsal angle such that a line through the mid-axis of the talus is generally in line with the first metatarsal shaft. In another embodiment, a subtalar implant is configured in the sinus tarsi to correct the AP-talar-first metatarsal angle such that a line through the mid-axis of the talus is generally in line or lateral to the first metatarsal shaft.

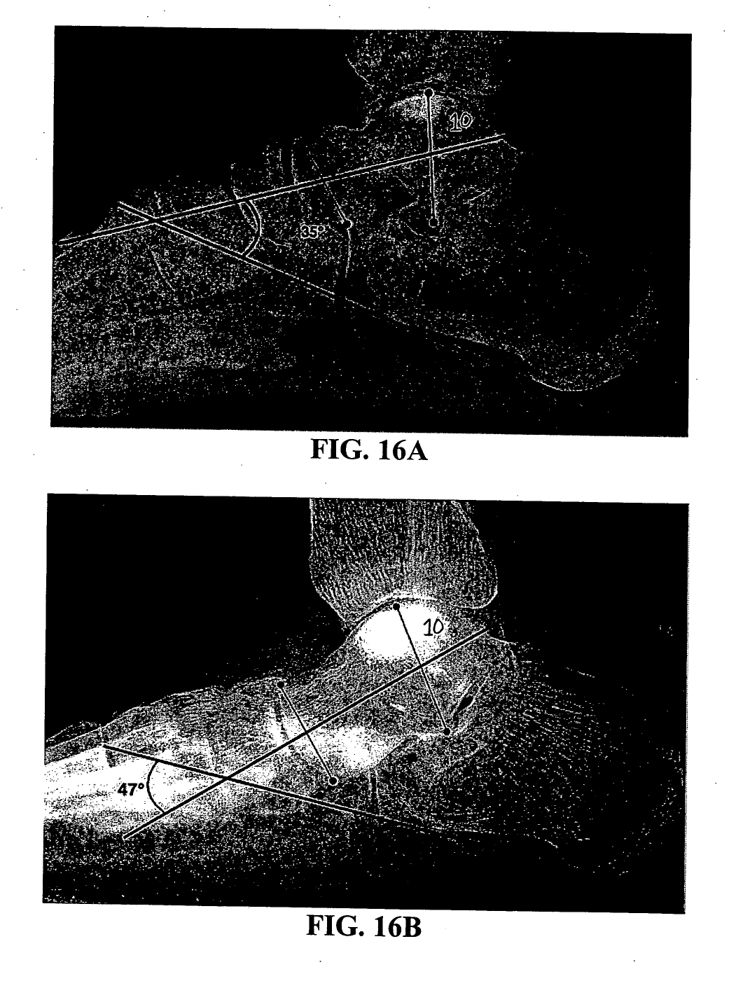

FIGS. 16A and 16B depict radiographs evaluating the lateral talocalcaneal angle. The lateral talocalcaneal angle is the angle formed by the intersection of a first line bisecting the talus 10 with a second line along the plantar border or through the long axis of the calcaneus 2. The first line is drawn through two midpoints in talus 10, one at the body and one at the neck. The angle is formed by the intersection of these axes. As shown in FIG. 16A, the normal range is about 25 to about 45 degrees. As depicted in FIG. 16B, an angle over about 45 degrees generally indicates hindfoot valgus, another component of pes planus. In one embodiment, a subtalar implant is configured in the sinus tarsi to correct the lateral talocalcaneal angle to about 15 degrees to about 60 degrees. In another embodiment, a subtalar implant is configured in the sinus tarsi to correct the lateral talocalcaneal angle to about 25 degrees to about 45 degrees. In a preferred embodiment, the lateral talocalcaneal angle is generally corrected to about 35 degrees.

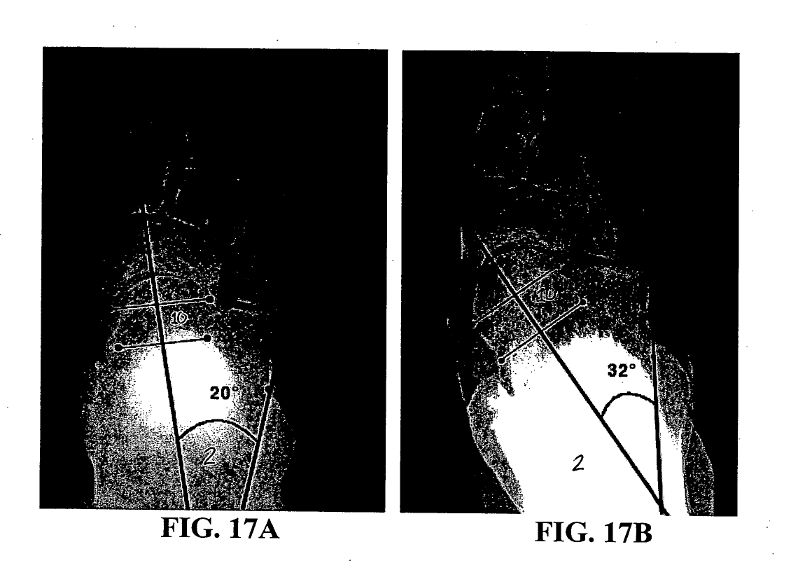

FIGS. 17A and 17B depict radiographs evaluating the AP talocalcaneal angle, also known as Kite's angle. This is the angle formed by the intersection of a line bisecting the head and neck of talus 10 and a line running parallel with the lateral surface of calcaneus 2. FIG. 17A depicts a foot within the range of normal for adults between about 15 degrees to about 30 degrees. Referring to FIG. 17B, an angle generally greater than about 30° indicates hindfoot valgus, another component of pes planus. In one embodiment, the subtalar implant is configured in the sinus tarsi to correct Kite's angle to about 50 degrees or less. In another embodiment, the subtalar implant is configured to correct Kite's angle to about 30 degrees or less. In still another embodiment, a subtalar implant is configured in the sinus tarsi to correct Kite's angle within a range of about 10 degrees to about 30 degrees.

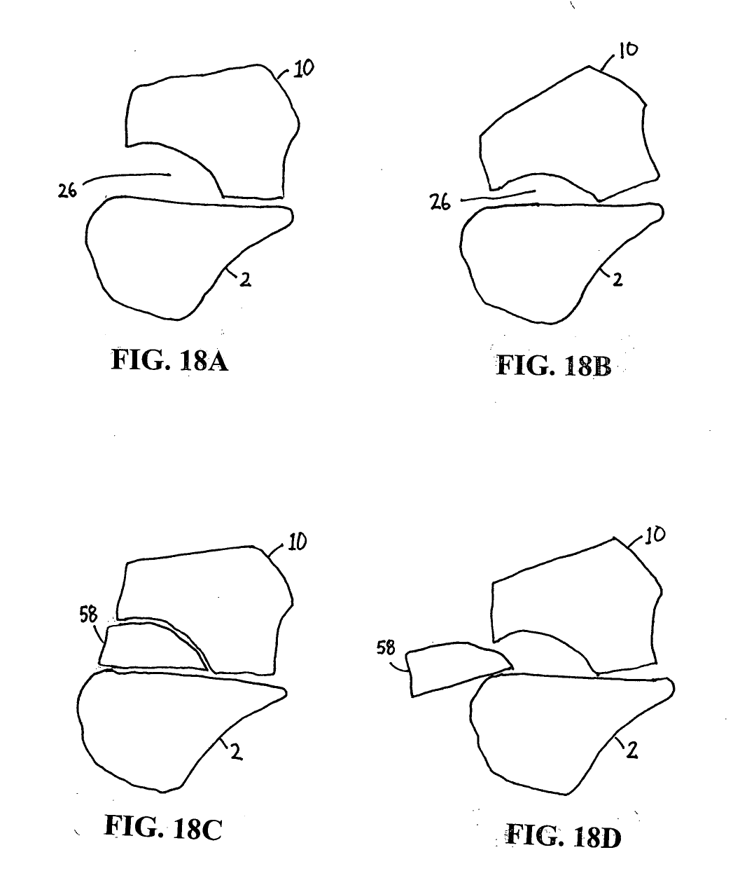

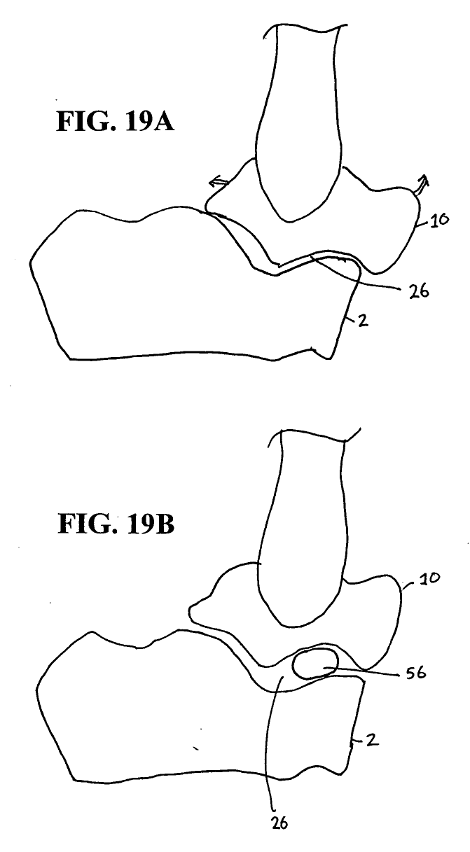

FIGS. 18A and 18B are schematic cross-sectional representations through the sinus tarsi of a neutrally aligned foot compared to a hyperpronated foot, respectively. Due to ligament laxity, the hyperpronated foot has a greater range of motion at talus 10 and calcaneus 2, which causes a shift in load bearing along the medial portion of the foot and tends to flatten the arch. Insertion of material 58 into sinus tarsi 26, alters the range of subtalar motion and limits the range of pronation. FIG. 18C shows that material 58 positioned in sinus tarsi 26 can have a wedge-type effect to position calcaneus 2 to a neutral alignment. FIG. 18D illustrates, however, that over time, the configuration of talus 10 and calcaneus 2 also has a tendency to cause lateral displacement of material 58 through forces exerted onto material inserted into sinus tarsi 26. FIGS. 19A and 19B are schematic longitudinal cross-sectional representations of a hyperpronated foot before and after insertion of material 58 into sinus tarsi 26.

Accordingly, one embodiment of the present invention provides an implant 60 which can be easily located within the tarsal canal, which may or may not deform under post-operative compressive forces, which would ensure that the desired amount of calcaneal eversion has been provided after insertion of the implant 60 and which can be imaged using radiography to determine whether the implant has been properly positioned during the procedure. By placing a device into the tarsal space between talus 10 and calcaneus 2, hindfoot motion and stability may be favorably modified. Such a device may further provide midfoot stability because midfoot-stability is co-dependent on hindfoot stability. Dysfunction of the posterior tibial tendon that supports the foot arch may also be treated by restoring the arch of the foot and relieving the excessive tension on the tendon.

By developing a minimally invasive, catheter-deliverable subtalar implant, disruption of the joint capsule and the ligamentous structures in and around the lateral portion of the foot can be reduced. Current subtalar implants require either transection of the ligaments overlying the sinus tarsi or the dilation of an opening up to about 3/4 inch diameter through the ligaments. Dilation of this magnitude will stretch and disrupt the ligaments. In general, the implant in accordance with the present invention may be advanced through a tissue opening of no greater than about 7 mm, and preferably no greater than about 2 mm to about 3 mm.

The development of an enlargeable implant will allow the implantation of an in-situ customized prosthesis that will also minimize trauma to the surrounding tissue during the implantation procedure and with long-term use. This will considerably shorten the postoperative recuperation period compared to existing devices and reduce postoperative pain and swelling. Moreover, because the integrity of the tissue overlying the sinus tarsi is preserved through minimally invasive implantation, the intact tissue is able to assist in anchoring the implant in the sinus tarsi. By customized, the inventor contemplates an implant that is at least partially conformable to the anatomical cavity in which it resides, at least prior to any polymerization or other curing step.

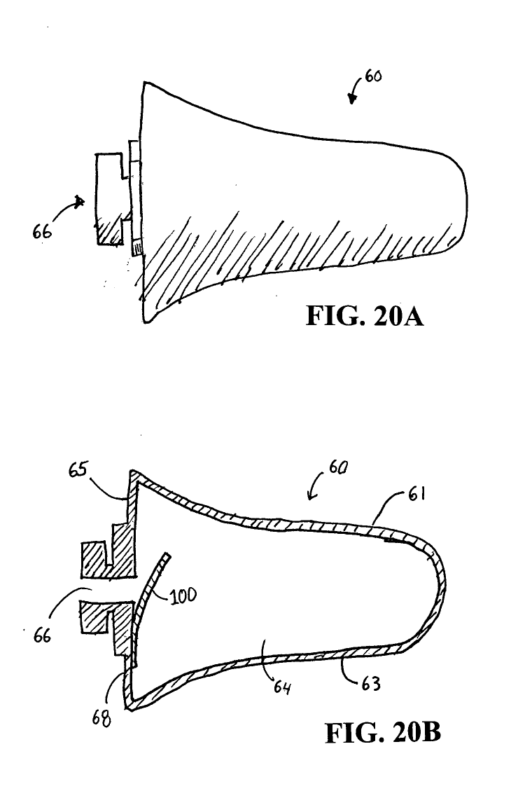

In one embodiment of the invention, illustrated in FIGS. 20A and 20B, the implant 60 comprises at least one inflatable compartment 64 and an inflation port 66. Inflation port 66 provides access to compartment 64 without compromising the integrity of compartment 64 and causing leakage. In one embodiment of the invention, implant 60 will inflate to a shape that approximates the shape of sinus tarsi 26. The shape of the sinus tarsi 26 is defined on its superior-medial surface 61 by the inferior surface of talus 10, on its inferior-medial surface 63 by the superior surface of calcaneus 2, and on its lateral surface 65 by soft tissue structures including the fascia. It is preferred, but not required, that the implant has a shape with an enlarged lateral surface 68. A large lateral surface takes advantage of the intact ligaments and soft tissue along the lateral border of sinus tarsi 26 to hold implant 60 in place. The lateral surface 68 has an area generally between about 2 square centimeters to about 5 square centimeters, preferably between about 3 square centimeters to about 4 square centimeters, and in one embodiment about 3.8 square centimeters.

A conformable implant 60 is also better adapted to affect the highly variable anatomy of the subtalar joint and to alter the highly variable geometry and motion of the joint. A conformable implant can be configured to have a greater contact surface area with sinus tarsi 26 and can disperse the loading of the subtalar joint across a greater surface area compared to non-conformable implants. The size and shape of sinus tarsi 26 is also varies with foot position. Therefore, the surgeon will position the foot during the procedure based upon the anatomy of a particular patient and the characteristics of the selected implant. One embodiment of the implantation procedure is described in detail below.

Generally, the area of the lateral-proximal surface 68 of the implant will be at least about twice the cross-sectional area of the dilated tissue access tract. Often, the lateral surface area will be at least 5×, 8×, 10× or 20× or more than the access tract to resist migration of the implant.

In another embodiment, the surgeon is able to limit certain dimensions or features of the implant by selecting a balloon having a shorter length, diameter and/or volume. The implant shape is further adjusted by allowing a variable degree of inflation. Variable inflation may allow deeper positioning of the implant within the sinus tarsi by providing implant 60 with a smaller diameter for deeper insertion into the narrow tarsal canal.

In still another embodiment, an implant having a predetermined shape is selected by the surgeon. The implant is compressible onto a catheter for minimally invasive delivery, but assumes a preconfigured shape with inflation. A preconfigured shape may be advantageously used to force a particular foot alignment or to facilitate anchoring of the implant. One indication for this implant and procedure is the hyperpronated, flexible and reducible flatfoot. The most common patient with this indication is pediatric, but adults with posterior tibial tendon dysfunction or hyper-pronation in the absence of subtalar joint and mid tarsal joint arthritis are also eligible.

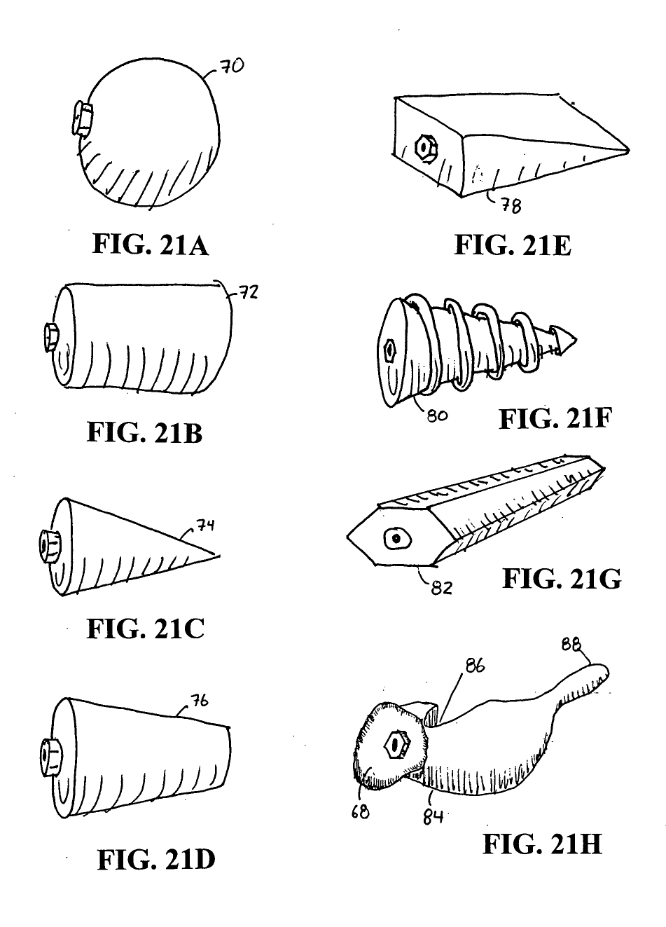

FIGS. 21A through 21H represent implants of various possible shapes for implants with predetermined shapes. The implant can be spherical 70, cylindrical 72, conical 74, frusta-conical 76, wedge-shaped 78, helical 80, polyhedral 82 or any three-dimensional shape 84 capable of positioning in the sinus tarsi. FIG. 21H is one embodiment of implant 60 advantageously fitted to the sinus tarsi 26 of a left foot. The implant, when inflated, may include a groove 86 or cavity dimensioned for fitting around the cervical ligament 40 and a distal tip 88 for anchoring implant 60 in a narrowing of the sinus tarsi 26 along the interosseous ligament 38. A large lateral surface area 68 uses the soft tissue at the lateral opening of the sinus tarsi 26 to maintain the desired position of the implant. This implant has a length of about fifteen millimeters to about twenty millimeters, a lateral diameter of about ten to about fifteen millimeters at the lateral end of the sinus tarsi and a medial diameter of about six to about eight millimeters at the medial end of the sinus tarsi.

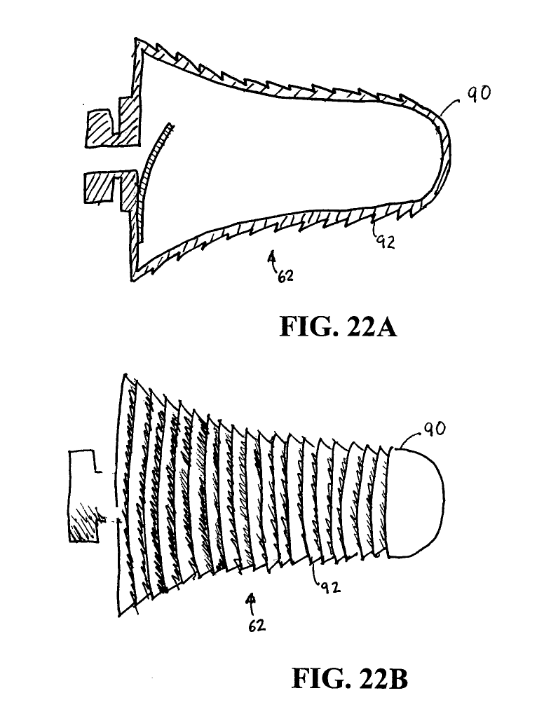

The outer surface 90 of implant 60 may be smooth, textured or comprise any of a variety of protrusions or indentations to cooperate with complementary anatomical structures to reduce the risk of implant migration. FIGS. 22A and 22B show one embodiment of the invention with a plurality of ridges 92 on the outer surface. Texturing on the outer surface 90 of implant 60 may provide an interference fit or increased friction between implant 60 and sinus tarsi 26 to resist displacement of implant 60 from its desired position. In a further embodiment, the outer surface 90 may further comprise one or two or more cellular ingrowth regions that allow ingrowth of the surrounding tissue and further resist displacement of the implant. The pore size of the cellular ingrowth regions may range from about 20 μm to about 100 μm or greater. Desirably, the porosity of the cellular ingrowth regions ranges from 20 μm to 50 μm and, in many embodiments, the porosity of the cellular ingrowth regions ranges from 20 μm to 30 μm.

If more aggressive anchoring of the implant is desired, attachment structures may be provided to facilitate attachment of implant 60 to soft tissue or bone. In one embodiment, sutures, clips, staples, tacks, pins, hooks, barbs, or other securing structures that can at least partially penetrate the surrounding tissue or bone are used. Depending on the location, length and other characteristics of the anchor on the implant and the anchor site within the sinus tarsi, the axis of movement of the subtalar joint may be further modified.

These securing structures may be made from any of a variety of materials, including metals, polymers, ceramics or absorbable materials. Absorbable materials include but are not limited to polylactic acid (PLA) or copolymers of PLA and glycolic acid, or polymers of p-dioxanone and 1,4-dioxepan-2-one. A variety of absorbable polyesters of hydroxycarboxylic acids may be used, such as polylactide, polyglycolide and copolymers of lactide and glycolide, as described in U.S. Pat. Nos. 3,636,956 and 3,297,033, which are hereby incorporated in their entirety herein by reference. The use of absorbable materials allows the securing structure to dissolve or resorb into human tissue after a known or establishable time range, from a week to over a year.

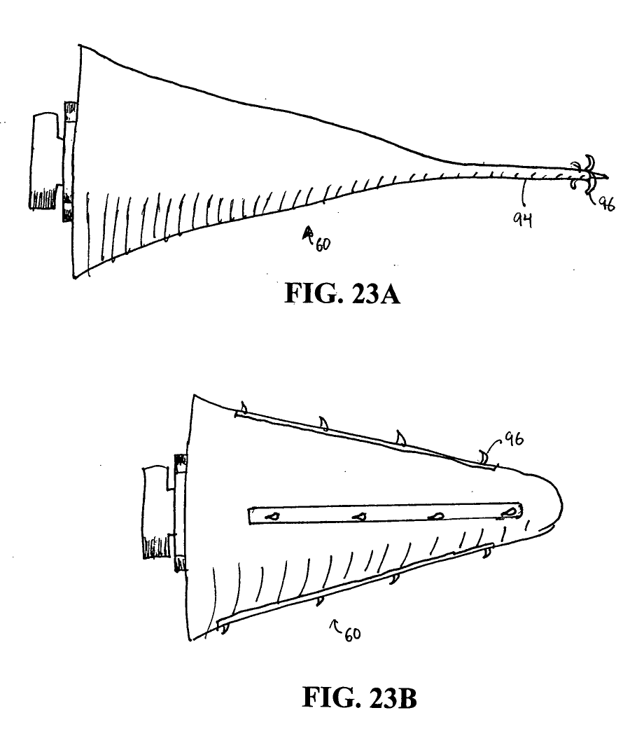

In one non-limiting example, shown in FIG. 23A, a distal anchor 94 with at least two or three or four or more barbs 96 is attached to the medial surface 98 of implant 60 for anchoring at the medial portion of the sinus tarsi 26. In another non-limiting example in FIG. 23B, one or more short pointed barbs 96 are integrally formed with implant 60 or secured thereto using any of a variety of attachment techniques which are suitable depending upon the composition of implant 60. As the implant is inserted into sinus tarsi 26, barbs 96 penetrate the surrounding soft tissue, bone or ligaments. Hooks may also be attached to or integrally formed with implant, so that the implant can be hooked into the surrounding tissue, possibly in combination with the use of a bioadhesive. Such hooks and barbs may be formed from a bioabsorbable or dissolvable material as has discussed above.

In another embodiment, the implant may come in contact with the leading edge of the posterior facet of the subtalar joint and the floor of the sinus tarsi. In this embodiment, the implant may be attached to the calcaneus by some means, and may alter the axis of movement of the subtalar joint by changing the way the talus and calcaneus interact relative to one another by extending the posterior facet and causing it to function around a different axis.

In one embodiment of the invention, implant 60 comprises any of a variety of flexible materials that resist stretching. These materials include but are not limited to polyethylene, polyolefins, polyvinyl chloride, polyester, polyimide, polyethylene terephthalate (PET), polyamides, nylon, polyurethane and other polymeric materials. One skilled in the art can select the material based upon the desired compliance, biocompatability, rated burst pressure and other desired characteristics. In one embodiment, the inflatable member has a wall thickness of about 0.001 cm to about 0.05 cm. In another embodiment, the inflatable member has a thickness of about 0.02 cm to about 0.03 cm.

Generally, the inflatable member has a rated burst pressure of greater than about 60 atmospheres (ATM) for resisting bursting and extrusion of inflation material under physiologic loading. In another embodiment, the inflatable member has a rated burst pressure of at least about eight ATM or more. A lower burst pressure can be used where a curable material is used to inflate the inflatable member and will bear the loading of the subtalar joint.

In a further embodiment of the invention, implant 60 is provided with one or more deformable wire supports within the material used to form the inflatable member. One possible function of the wire support to provide some stiffness to the implant during the insertion process to allow the operator to insert the implant into distal sulci or crevices of the sinus tarsi. A wire support can comprise a shape memory metal, such as nitinol. Upon insertion of the implant into the sinus tarsi, the body heat of the patient will cause the wire support to change shape and expand to the borders of the sinus tarsi. Those skilled in the art understand that any of a variety of biocompatible, deformable metals or rigid polymers may be used to form the skeleton.

In addition to providing access to inflate the inflatable compartment, the inflation port 66 may comprise other features to facilitate use of the implant. The inflation port may be self-sealing or have a one-way valve to obviate the need for a separate sealing of the implant after inflation. Valve configurations include but not limited to hemostatic-type valves, flap valves or duckbill valves. In some embodiments, a pierceable septum may be used. A flap valve 100 is shown in FIG. 20B. The flapper portion of the valve can be formed from silicone, rubber, neoprene or any of a variety of other flexible materials known to those with skill in the art. Less flexible materials may be used for the valve where the inflation fluid is highly viscous or curable. One skilled in the art can select the type of seal based upon the inflation pressures of the implant, the viscosity of the inflation fluid, curability and other characteristics. Inflation port 66 may be further configured to minimize any leakage of material from either implant 60 or the delivery system during the detachment process. Inflation port 66 may include radio-opaque markers to allow a clinician to later deflate or adjust implant 60 transcutaneously with a hypodermic needle.

The inflation media used to inflate inflatable compartment 62 may include any of a variety of biocompatible materials, including but not limited to saline, silicone polymers, polyurethane polymers, linear or branched polyols, PMMA or others known in the art. Solid materials, such as small polymeric metallic microspheres, microtubules or microdiscs can also be used as a filling agent. The material can also be a combination of materials, such a curable liquid substrate and a catalyst, that can solidify within implant 60. Several U.S. patents disclose various types of polymers or proteins that, assertedly, can be injected into a joint as a liquid or semi-liquid composition that subsequently harden into a solidified material. For example, U.S. Pat. No. 5,556,429 (Felt 1996), herein incorporated by reference, discloses injection of a fluidized mixture of a biocompatible polymer (such as a silicone or polyurethane polymer) and a biocompatible “hydrogel” (a hydrophilic polymer, formed by steps such as using an agent such as ethylene dimethacrylate to cross-link a monomer containing a hydroxyalkyl acrylate or methacrylate), into a space. After injection, the polymer and hydrogel mixture can be set into solidified form by means such as ultraviolet radiation, which can be introduced into the space by a fiber optic device. Other combinations of inflation materials may include the addition of iodine, barium or other radio-opaque component. One skilled in the art can select the material based upon the desired viscosity, density, cure time, degree of exothermic cure reaction, radio-opacity and other characteristics. For curable materials, one skilled in the art may consider the load-bearing strength, tensile strength, shear strength, fatigue, impact absorption, wear characteristics and other factors of the cured material.

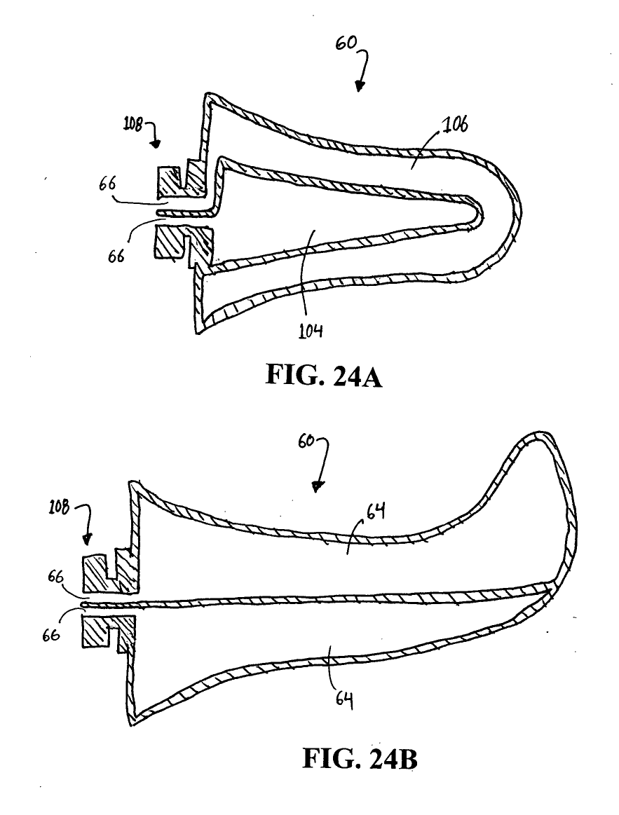

In another embodiment, implant 60 has multiple inflation ports and multiple compartments such that different portions of implant 60 can be independently inflated. FIGS. 24A and 24B are non-limiting examples of two-compartment inflatable members. The delivery catheter for an implant comprising multiple compartments may have multiple inflation lumens, each with a unique port to allow independent inflation of the compartments. Different compartments may be inflated with different materials and/or different pressures, to produce different multizone characteristics. In one embodiment of the invention, implant 60 has an inner compartment 104 at least partially encapsulated by an outer compartment 106. Outer compartment 106 may be inflated with a curable material to provide a solid material at the surface of implant 60. Inner compartment 104 may be inflated with a liquid material to provide limited deformability to implant 60. Alternatively, outer compartment 106 may be inflated with a liquid material and inner compartment 104 is inflated with a curable material. This particular embodiment may provide cushioning to the joint surfaces by providing a compressible implant surface, yet the curable core provides some resistance to complete compression.

Implant 60 further comprises a coupling interface 108 that releasably attaches implant 60 to the delivery system. Coupling interface 108 is generally located on or about inflation port 66 and allows for inflation of implant 60 through the delivery system without leakage of material into the surrounding tissue. Coupling interface 108 also allows transmission of force, including torque, from the delivery system to the implant to facilitate positioning of implant 60. Coupling interface 108 is configured to allow detachment of implant 60 from the delivery system and, optionally reattachment of the delivery system, such as to permit reinflation, repositioning or removal

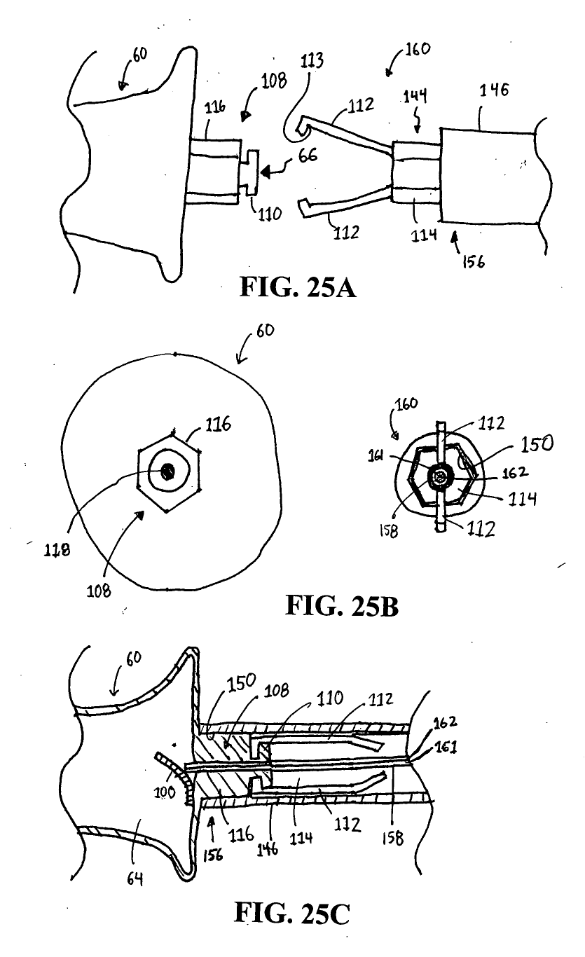

FIGS. 25A through 25C illustrate a releasable connection in accordance with the invention, where coupling interface 108 is releasably retained by a deployment catheter. Coupling interface 108 carries an engagement surface such as the distal surface of a flange 110 surrounding inflation port 66. Flange 110 is capable of being grasped by prongs 112 extending from the delivery catheter 114. Coupling interface 108 further comprises a base 116 having a polygonal or otherwise rotationally keyed cross-section. Base 116 may be positioned between coupling interface 108 and inflatable compartment 64 and is capable of forming another rotationally engaged mechanical interfit with an outer sheath 146 over catheter 114. This additional mechanical interfit provides further resistance to dislodging or separation of implant 60 from delivery catheter 114 during implantation, especially from rotational forces.

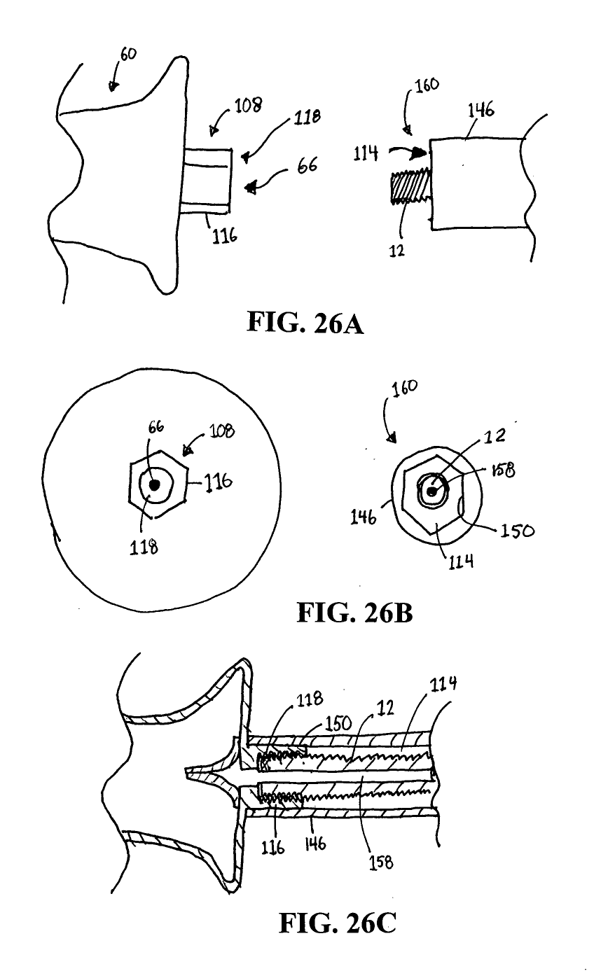

FIGS. 26A through 26C depict another embodiment of coupling interface 108, comprising base 116 and an internal threaded lumen 118 for accepting a threaded core 120 extending from the delivery catheter 114. The attachment of coupling interface 108 to delivery catheter 114 is described in further detail below.

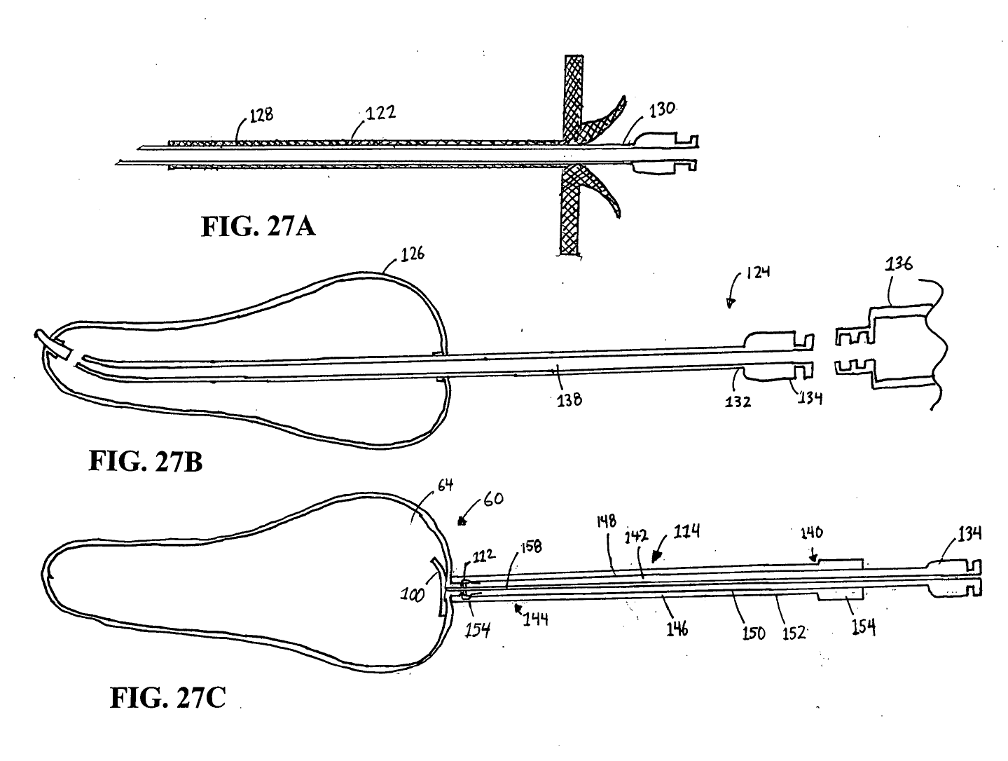

One embodiment of the delivery system is illustrated in FIGS. 27A through 27C, comprising a cannula or sheath 122, a sizing catheter 124 with an inflatable balloon tip 126 and delivery catheter 114 attachable to implant 60. Cannula 122 is made from plastic with radio-opaque markers to allow imaging of the cannula. Cannula 122 can be introduced into the sinus tarsi over a needle 130. Cannula 122 has a length of about two inches to about six inches and a diameter of about 12 gauge to about 18 gauge. A lumen 128 is provided in cannula 122 to allow passage of sizing catheter 124 and delivery catheter 114 with attached implant 60. Alternatively, the cannula can be made of metal and has a distal tip sufficiently sharp to pierce the skin, connective tissue and ligaments overlying the sinus tarsi. A metal cannula with a sharp tip may not require insertion of the cannula over a needle or guidewire.

Sizing catheter 124, shown in FIG. 27B, has a length of about two inches to about eight inches and a diameter capable of passing through cannula 122. Sizing catheter 124 has radiographic markers for determining its position in the foot during radiographic imaging. The proximal end 132 of sizing catheter 124 comprises a Luer fitting 134 or other similar type of mechanical lock for attaching a syringe 136. A lumen 138 within the sizing catheter 124 provides a conduit from syringe 136 to sizing balloon tip 126 at the distal end of sizing catheter 124. Sizing balloon tip 126 generally has a length of about fifteen millimeters and an inflated diameter of about six to about twelve millimeters. Sizing balloon tip 126 can have any of a variety of shapes similar to those described for implant 60. Syringe 136 has markings so that the volume of fluid inflated into sizing balloon tip 126 can be measured quantitatively. Sizing catheter 124 is capable of performing a number of functions. Insertion of sizing catheter 124 through cannula 122 initiates the dilatation of sinus tarsi 26 and helps to prepare the path for introduction of permanent implant 60. By filling sizing catheter balloon 126, the surgeon is able to determine the volume of non-compressible fluid required to fill the implant 60 to achieve the desired post-implantation pronation.

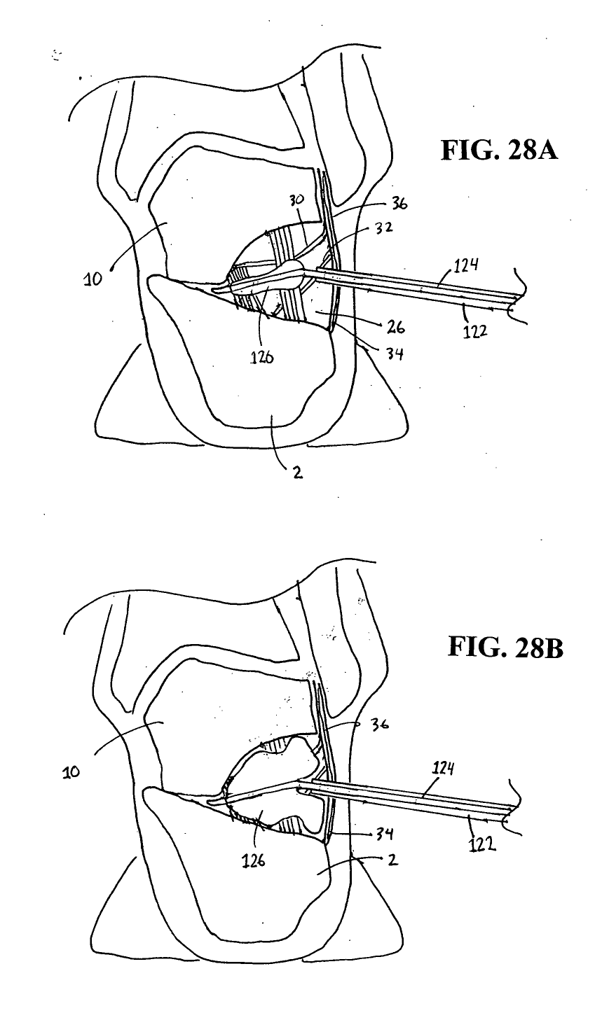

Sizing balloon 126 may comprise a high-compliance material that is capable of conforming to the surrounding anatomical structures. By filling sizing balloon 126 with a radio-opaque fluid under fluoroscopy or with radiography, the surgeon can determine the proper three-dimensional shape of the cavity 26. An implant 60 can then be selected to correspond with the predetermined shape and/or size. FIG. 28A is a cross-sectional schematic view of a sizing catheter 124 with an uninflated high-compliance sizing balloon 126 in sinus tarsi 26. As the balloon 126 is inflated in FIG. 28B, loose ligaments and connective tissue will be displaced as balloon 126 conforms around taut structures. Visualization of this shape information permits selection or construction of an implant having a predetermined shape or determination of the need for a semi-customizable or fully customizable implant.

In an alternative embodiment of the delivery system, sizing catheter 124 is omitted because the inflation characteristics of the implant allow implant 60 to be adapted to structural variations of the anatomy. Selection of a particular size or shape of implant is not required in this alternative embodiment. In this embodiment, the surgeon can partially inflate the implant, evaluate the effect on the foot alignment and flexibility, and continue to inflate, deflate and/or position the implant until a desired displacement, alignment or range of motion limiting result is achieved. The delivery catheter 114 may then be detached and withdrawn, leaving the implant 60 in place.

FIG. 27C shows one embodiment of delivery catheter 114, comprising a proximal end 140, a body 142, a distal end 144 and an outer sheath 146. The delivery catheter has a length of about two inches to about ten inches and has a diameter capable of passing through cannula 122. Catheter 114 may contain radiographic markers for determining its position in the foot with imaging. Proximal end 140 of delivery catheter 114 comprises at least one Luer fitting 134 or other similar type of mechanical lock for attaching a syringe to inflate the implant with inflation media. Body 142 of delivery catheter 114 comprises at least one lumen 148 to provide a conduit from the syringe or other source to implant 60 fastened to distal end 144 of delivery catheter 114. A multi-lumen catheter may be used where the implant has multiple compartments, or where multiple reactive materials are used to inflate the implant. The use of multiple lumens may prevent reactive components of the implant material from reacting within the catheter and prevent clogging of the catheter. For inflation materials that use ultra-violet light for curing, a fiber-optic line can be inserted through the lumen 148 to provide the ultra-violet light. Outer sheath 146 comprises an inner surface 150, an outer surface 152, a proximal portion 154 and a distal portion 156. Outer sheath 146 also has a retracted position that exposes the distal end 144 of delivery catheter 114 and an extended position that covers distal end 144 of delivery catheter 114.

Distal end 144 of delivery catheter 114 comprises an inflation lumen 158 and a coupler for attaching to coupling interface 108 of implant 60. In the embodiment of the invention seen in FIG. 25A, where coupling interface 108 comprises flange 110, the coupler 160 of delivery catheter 114 comprises a plurality of radially outward-biased or movable graspers or prongs 112 extending distally to an engagement surface. Graspers 112 may comprise bent wires, thin arcuate sheets, or any other configuration known to those with skill in the art that is capable of engaging flange 110 and applying a proximally directed force to flange 110.

Referring back to FIG. 27C, when outer sheath 146 of delivery catheter 114 is in the distally extended position, inner surface 150 of outer sheath 146 will contact prongs 112 and apply radially inward forces against prongs 112. These forces move the prongs 112 closer together and allow the engagement surfaces 113 of prongs 112 to engage the complementary engagement surface on flange 110 of implant 60.

In FIG. 25C, if outer sheath 146 is further distally extended, inner surface 150 of sheath 146 will contact base 116 of coupling interface 108. Base 116 of implant 60 has a polygonal cross-section capable of forming a mechanical anti-rotation interfit with a polygonal cross-section of inner surface 150 of outer sheath 146. Distal portion 156 of sheath 146 will also exert a distally directed counterforce on implant 60 in opposition to the proximally directed force on the implant from the prongs 112 to firmly attach implant 60 to the delivery catheter 114. If sheath 146 is retracted, the mechanical interfit with base 116 is relieved and radially inward forces on prongs 112 are removed. Prongs 112 will resume their outward bias and distract from flange 110 of implant 60, causing release of implant 60. As previously mentioned, FIG. 25C illustrates that delivery catheter 114 may optionally comprise a slideable inner core 161 within the inflation lumen 158 of delivery catheter 114 that is capable of extending through coupling interface 108 to engage inflation port 66 of implant 60. A lumen 162 in slideable core 161 provides a conduit to inflate attached implant 60 with inflation media.

In the embodiment of implant 60 shown in FIG. 26A, where coupling interface 108 comprises a threaded lumen 118, the delivery catheter 114 comprises an outer sheath similar to the sheath described above. The inner core of this embodiment of the delivery catheter, however, comprises a threaded inner core 120 with lumen 158, where threaded core 120 is complementary to threaded lumen 118 of implant 60. Implant 60 attaches to delivery catheter 114 by rotating threaded core 120 into threaded lumen 118 of the implant. To resist rotation of implant 60 from frictional forces during the attachment or detachment of implant 60, the polygonal cross-section of inner surface 150 of outer sheath 146 is capable of forming an anti-rotational mechanical interfit with the polygonal cross-section of coupling base 116 on implant 60 when outer sheath 146 is extended.

In an alternative embodiment of the delivery system, a guidewire or guide pin having a diameter of about 0.010 inch to about 0.038 inch and a length of about four inches to about eight inches is provided for insertion into the sinus tarsi. The guidewire is insertable through a needle inserted into the sinus tarsi. The needle is withdrawn after the guidewire is positioned. An introducer may be passed over the guidewire to further dilate the passage to the sinus tarsi. The sizing and delivery catheters are adapted for passage over the guidewire into the sinus tarsi. In this embodiment, both catheters would each have at least two lumens. One lumen is used to pass the catheter over the guidewire and the other lumen would be used to inflate the sizing balloon or implant. These lumens may be oriented in a dual concentric configuration or adjacent to each other.

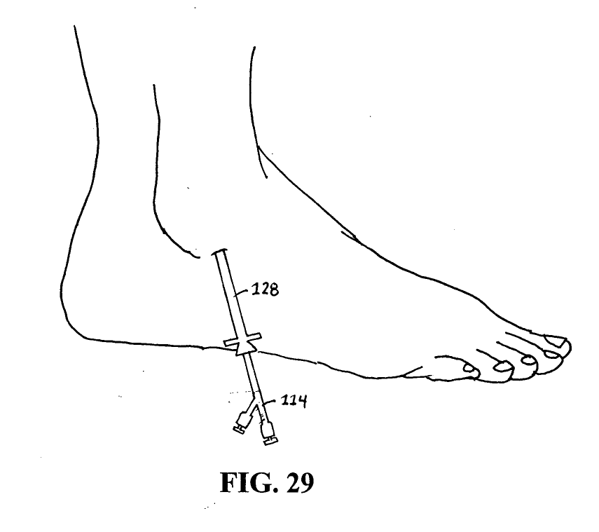

One indication for this embodiment of the implant and implantation procedure is a reducible, hyperpronated, flexible flatfoot. These patients are commonly pediatric, but adults with posterior tibial tendon dysfunction and/or hyper-pronation in the absence of subtalar joint and mid tarsal joint arthritis are also potential candidates. FIG. 29 shows one procedure for using an embodiment of the implant comprises positioning the patient on a table and draping the lateral side of the foot in the usual sterile fashion known in the art. The insertion site for the implant is identified by palpation of bony markers, including but not limited to the fibular head, cuboid, talus and calcaneus bones. The lateral opening of the sinus tarsi is identified anterior, medial and inferior to the lateral malleolus or distal head of the fibula. Local anesthesia is injected into the skin and the connective tissue overlying the insertion site. Anesthetics with epinephrine may be used to limit bleeding at the insertion site. Alternatively, regional or general anesthesia may be used.

The surgeon places the foot in a slightly supinated position to widen the lateral opening of the sinus tarsi during the procedure. A needle is inserted at the desired site and a small cannula is passed over the needle. The desired depth of insertion is determined by markings on the cannula and assisted by fluoroscopic imaging. The needle is then withdrawn. The cannula may be of “peel-away” type as is known to those with skill in the art.

The foot with the inserted cannula is radiographically imaged to facilitate positioning of the cannula in the sinus tarsi. FIG. 28A illustrates sizing catheter 124 with an attached, fluid-filled syringe inserted through cannula 122. The foot is then repositioned and held in a generally neutral alignment. Neutral alignment is defined as the foot position where the lateral aspect of the heel becomes perpendicular to the leg and the talonavicular joint feels congruous to palpation. Neutral alignment is often, but not always, the position in the range of motion where the foot is capable of two-thirds additional supination and one-third additional pronation. Foot alignment can also be checked radiographically by assessing changes to the cyma lines in the AP and lateral views of the foot, as previously shown in FIGS. 9A and 10A.

Referring to FIG. 28B, balloon tip 126 on sizing catheter 124 is inflated until significant resistance is met. The inflation volume on the syringe is measured. The surgeon assesses the range of motion and alignment of the foot with the inflated sizing catheter in place. This allows the surgeon to estimate the potential changes to the joint and to facilitate selection of the permanent implant. The surgeon also checks the quality, range, location and smoothness of joint motion. Radiographic imaging may be performed for additional assessment of the joint. The cannula is repositioned and/or the sizing balloon volume is adjusted to achieve a desired degree of foot eversion (e.g. approximately four degrees). As noted previously, approximately one third of the subtalar range should be in the direction of pronation and two-thirds towards supination. Balloon tip 126 is deflated and sizing catheter 124 is withdrawn.

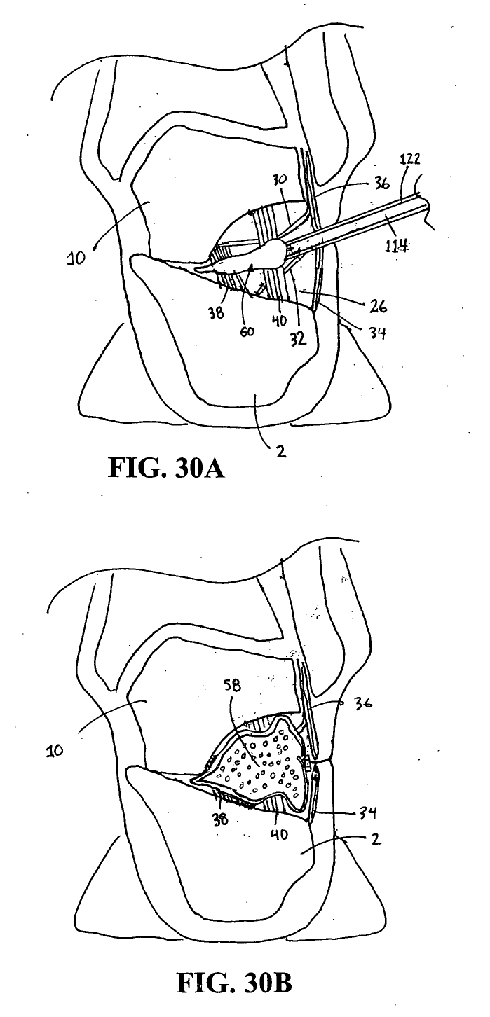

FIG. 30A shows delivery catheter 114 with selected inflatable implant 60 passed through cannula 122 and into sinus tarsi 26. Cannula 122 is optionally peeled away from the foot. Implant 60 is inflated with at least one inflation medium 58 to the desired volume based upon the inflation volume measured with sizing catheter 124. Foot alignment and range of motion is rechecked by physical exam and/or radiographic imaging. The inflation volume of implant 60 may be adjusted based upon the results of the exam and/or the imaging until the desired talocalcaneal position is achieved. In one embodiment, the surgeon uses the cyma line, in contradistinction to an anterior displaced talonavicular joint, as an indication that a pronated foot has been reduced to a more neutral alignment. Implant 60 is then sealed, if implant 60 is not self-sealing. Referring to FIG. 30B, delivery catheter 114 is detached from implant 60 and both catheter 114 and cannula 122 are withdrawn from the patient. If necessary, the insertion site is closed by either suturing or adhesives and dressed. A splint or cast is applied to the foot.

In an alternative implantation procedure, the material used to inflate implant 60 to the desired volume is removed from the implant and its volume is measured. An equal or similar volume of another material having a different density or characteristics is used to reinflate the implant. This alternative procedure may be used to obtain a more accurate measurement of the sinus tarsi and the volume of final inflation material to be used where the final inflation material changes volume as it cures. The volume of the initial fluid used to assess the sinus tarsi is used to calculate the volume of uncured final inflation material to be delivered.

In another alternate method of implanting the device using a guidewire, the patient is placed on a table and the lateral side of the foot is draped in the usual sterile fashion known in the art. The insertion site for the device is identified by palpation of bony markers, including but not limited to the fibular head, cuboid, talus and calcaneus bones. Local anesthesia is injected into the skin and connective tissue overlying the insertion site. Anesthetics with epinephrine may be used to limit bleeding at the insertion site. A large bore needle is inserted at the desired site and a guidewire is passed through the needle. Optionally, a small dilator is passed over the guidewire for enlarging the pathway to the sinus tarsi. The foot with the inserted guidewire is radiographically imaged to confirm positioning of the guidewire in the sinus tarsi.

A catheter with the inflatable implant at the catheter tip is passed over the guidewire and into the sinus tarsi. The implant is inflated to the desired volume. The talo-calcaneal relationship is checked by physical exam and/or radiographic imaging. The inflation volume of the implant may be adjusted based upon the results of the exam and/or the imaging until the desired talo-calcaneal position is achieved. The surgeon may use the cyma line, in contradistinction to an anterior displaced talo-navicular joint, as an indication that a pronated foot has been reduced to a more neutral alignment. The delivery catheter is detached from the implant and both the catheter and guidewire are withdrawn from the patient. The insertion site is closed by either suturing or adhesives and dressed.

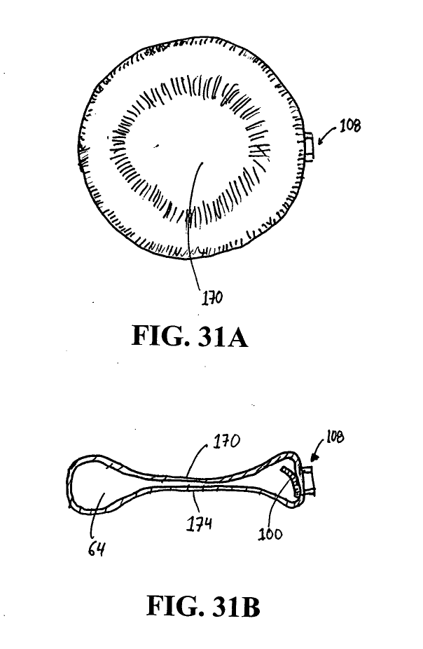

The implant and delivery system described above can also be adapted for insertion into the first MTP joint of the foot. Referring to FIGS. 31A and 31B, the implant shape for this embodiment of the invention is preferably an implant comprising a first concave surface 170 on a first side of the implant 172 and a second concave surface 174 on a second side. First concave surface 170 is adapted to contact the distal end of the first metatarsal and second concave surface 176 is adapted to contact the proximal end of the first proximal phalanx of the foot. Other shapes, however, can be used depending upon the particular anatomy and disease of the first MTP joint. The delivery system will generally have a shorter length because of the accessibility of the first MTP joint.

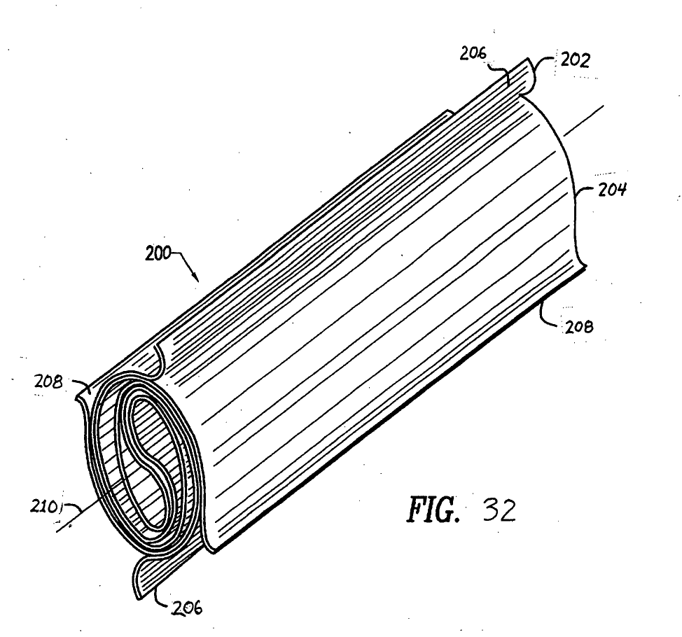

FIG. 32 is a schematic, isometric representation of one embodiment of the invention, comprising a rolled subtalar implant 200. Implant 200 may be self-expandable or expandable through the application of external force, such as a balloon catheter. The balloon catheter may be removed after the expansion of the implant 200, or the balloon may be detached and left within the sinus tarsi to further support the implant 200. Implant 200 may be constructed of two sheets 202 and 204 of resilient, biocompatible material, preferably a superelastic material or a shape memory material, as is known in the art. Nitinol is preferred, but in other embodiments, the implant may be constructed from another biocompatible metal, such as titanium, or a plastic or polymer material.

Sheets 202 and 204 are initially rolled tightly together into a cylindrical form. Each sheet of this compacted form is tightly rolled and implant 200 is inserted, in this compacted form, into the sinus tarsi of a foot, as described below. When the implant is then released inside the sinus tarsi, the resilience of sheets 202 and 204 causes them to partially unroll into an expanded state, so that implant 200 expands radially outward to assume an increased diameter, as shown in FIG. 32.

Preferably, outer edges 206 and 208 of sheets 202 and 204, respectively, are formed so that when implant 200 is released inside the sinus tarsi, the edges bend radially outward, as shown in FIG. 32. Edges 206 and 208 will then engage an inner surface of a bone surrounding the sinus tarsi, so as to hold implant 20 firmly in place and prevent sliding or rotation of the implant. Preferably, edge 206 is bent at an acute angle, and edge 208 is bent at an oblique angle, as shown in the FIG. 32, so that implant 200 resists rotation in both clockwise and counterclockwise directions about its axis 210.

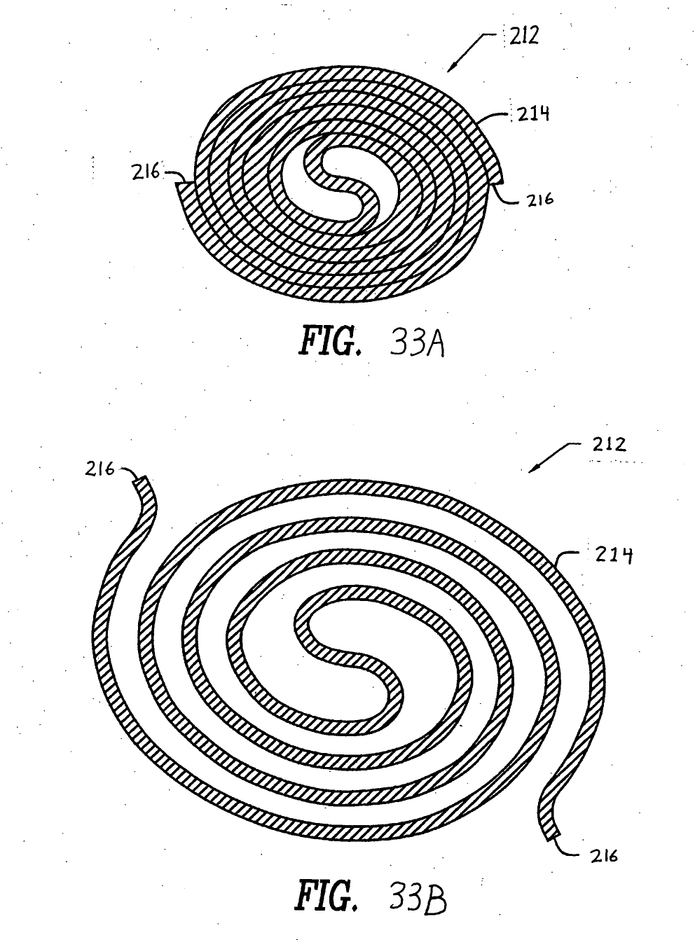

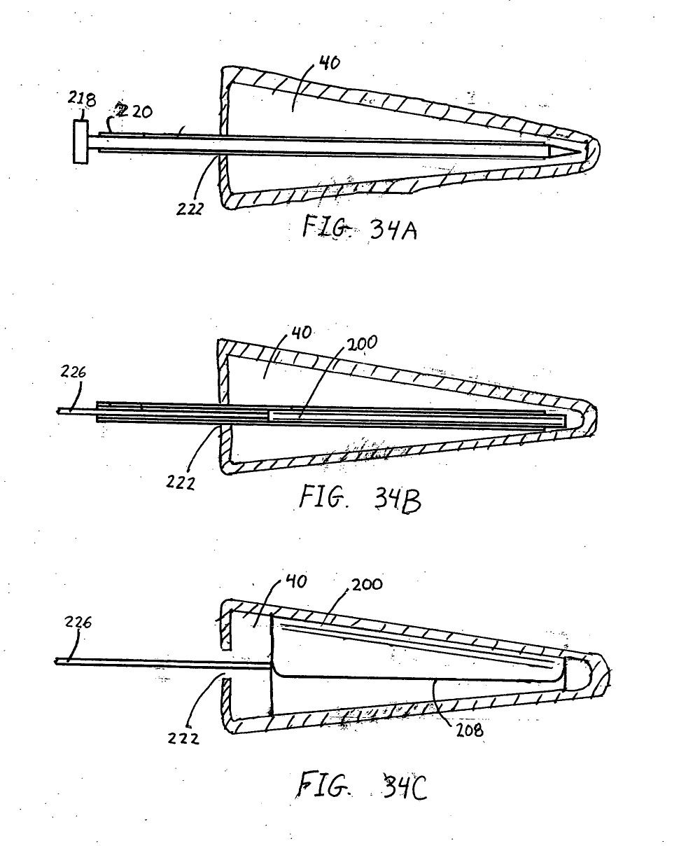

FIGS. 33A and 33B are schematic, sectional representations of a self-expanding implant 212, similar to implant 200, illustrating the principle of radial self-expansion of such implants. For simplicity of illustration, self-expanding implant 212 comprises only a single sheet 214 of self-expanding material, preferably resilient material. It will be understood by those skilled in the art that subtalar implants, as exemplified by implants 200 and 212, may comprise one, two or more sheets of self-expanding material, rolled together as shown in FIGS. 32, 33A and 33B.

FIG. 33A shows implant 212 in a first, closed configuration, in which the implant is compressed radially inward to facilitate its insertion into the sinus tarsi of a foot, as described below. In one embodiment, implant 212 has an outer diameter of less than about 4 mm in this closed configuration. In another embodiment, implant 212 preferably has an outer diameter of about 2 mm. FIG. 33B shows implant 212 in a second, open configuration, which the implant assumes after location within the cavity to fixate the bone. Preferably, the diameter of implant 212, in the open configuration of FIG. 33B, is at least 100% greater than the diameter in the closed configuration of FIG. 33A. More preferably, the diameter in the open configuration is at least about 300% the diameter in the closed configuration. The large diameter difference between closed and open configurations is advantageous in that it facilitates insertion of implant 212 into the foot in the closed configuration through a insertion site of minimal size made at lateral surface of the foot.

As described above with reference to implant 200, sheet 214 preferably comprises a superelastic material, preferably Nitinol, having a thickness selected to achieve the desired radial force, such as about 0.2 mm. The superelasticity of sheet 214 causes implant 212 to expand until outer edges 216 of the sheet engage the inner bone surface surrounding the sinus tarsi, to resist inward radial compression force from the surrounding bone.

Sheet 214 may comprise shape memory material, such as Nitinol, which is produced, as is known in the art, so as to have the open form shown in FIG. 33B and to be normally in the austenitic state at body temperature. In the closed configuration shown in FIG. 33A, however, the force exerted in rolling up sheet 38 preferably causes the material to assume a state of stress-induced martensite. In this state, the material is relatively flexible and elastic, making it easier to insert implant 212 into the foot. Once the implant has expanded inside the foot to the open configuration shown in FIG. 33B, however, the stress on sheet 38 is reduced, and the material reverts to its normal, substantially rigid austenitic state. The rigidity of the material in this state facilitates arthroereisis of the foot.

Additionally or alternatively, the shape memory material may have a critical temperature in the range between room temperature and body temperature, preferably around 30 degrees Celsius. As described above, the shape memory material is formed so that in its austenitic state (i.e. above the critical temperature), it has substantially the open, expanded form shown in FIG. 33B. Below the critical temperature, i.e. before insertion of implant 212 into the foot, the shape memory material is in a martensitic state, in which it is relatively flexible and elastic and is compressed into the closed configuration shown in FIG. 33A. When the implant is inserted into the foot, it is warmed (e.g. by body heat) to above the critical temperature, whereupon it opens and assumes its substantially rigid, austenitic state. Optionally, a heating element may be brought into contact with the implant once it is inside the foot, for example, as illustrated in FIG. 34B and described below, to hasten its expansion and state change.

FIGS. 34A to 34C are schematic, sectional illustrations showing the insertion of an implant 200 into sinus tarsi 40 of a foot. Although described with reference to the sinus tarsi, it will be appreciated that devices and methods in accordance with the present invention may be applied in other joints of the foot (e.g. the 1st MTP joint), with appropriate adaptations for the differences in size and mechanical strength required of the foot joints, as will be apparent to one of ordinary skill in the art.

As shown in FIG. 34A, a stylette 218 is inserted into a sinus tarsi within a cannula 220. Cannula 220 preferably comprises a syringe needle or catheter. Stylette 218 and cannula 220 are then introduced percutaneously into sinus tarsi through an opening 222 of the foot.

Alternatively, a small incision may be made through the skin and soft tissues, to visualize the sinus tarsi, and a passage may be formed in the sinus tarsi using blunt dissection for insertion of the cannula therethrough.

As shown in FIG. 34B, once cannula 220 is properly in place, stylette 218 is withdrawn, and implant 200, in its compressed, closed configuration, is passed into the lumen 224 of the cannula. Preferably, a plunger 226 is used to push the implant into the needle or catheter and hold it in place. Cannula 220 is then fully withdrawn, leaving implant 200 in the sinus tarsi.

Implant 200 expands or is expanded (e.g. by balloon dilatation) to substantially fill the sinus tarsi, as shown in FIG. 34C. The implant self-expands in the self-expandable embodiments disclosed herein. Alternatively, in other embodiments, as disclosed below, the implant is expanded using external force or energy. As mentioned previously, expansion of the implant may be performed with a balloon catheter. The balloon catheter may be removed from the foot after expansion of the implant, or alternatively the balloon may be detached and left in the foot to further support the implant.

The self-expansion of the implant forces curved edges 206 and 208 of sheets 202 and 204 (or 216 of implant 212) radially outward against the bones of sinus tarsi 40. This force anchors the implant in place and alters the alignment of the talus and calcaneus. In some preferred embodiments of the present invention, wherein sheets 202 and 204 comprise shape memory material as described above, plunger 226 may optionally comprise a heating element for heating implant 200 to above the critical temperature.

After implant 200 is positioned and anchored firmly in place, plunger 226 is withdrawn through the incision site, and the skin wound made by or for cannula 220 is allowed to close. Within a short time after completion of the procedure illustrated in the figures, the subject is able to mobilize the foot. The mechanical strength of implant 200 also reinforces the bone against axial and lateral forces that may be exerted on the foot.

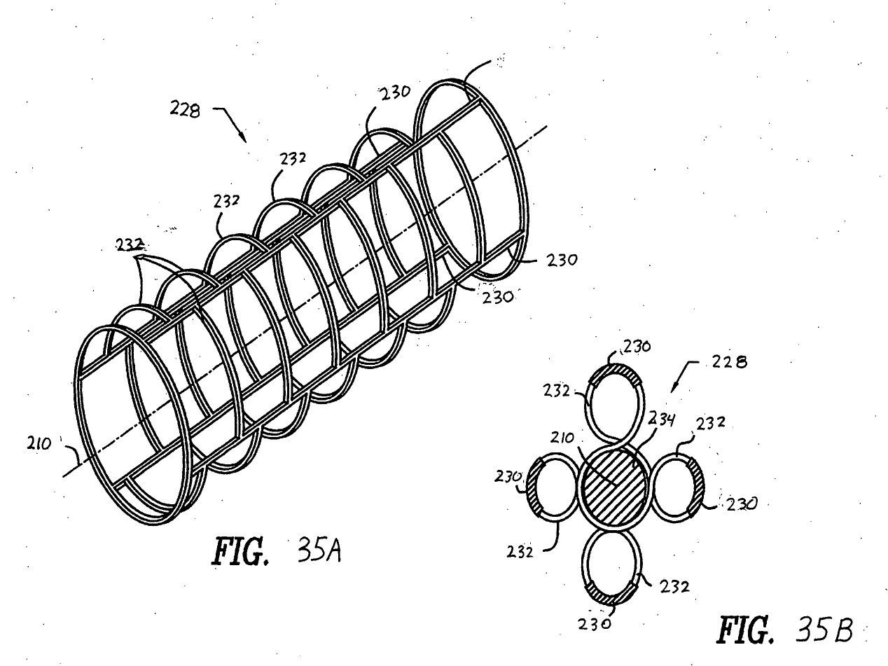

FIG. 35A is a schematic, isometric view of another self-expanding subtalar implant 228. Implant 228 comprises a plurality of longitudinal ribs 230, connected by a plurality of circumferential struts 232. Ribs 230 and struts 232 preferably comprise resilient material, preferably superelastic material, or alternatively, shape memory material as described above. FIG. 35A shows implant 228 in a substantially open configuration, which the implant assumes when it is located inside the sinus tarsi and allowed to expand.

FIG. 35B is a schematic, sectional illustration, showing implant 228 in a closed or constricted configuration for insertion of the implant into the foot. To compress the implant into this closed configuration, a long, cylindrical holding pin 234 (seen in sectional view in FIG. 35B) is inserted gradually along central axis 210 of the implant. As pin 234 is inserted, each circumferential strut 232 is, in turn, bent inward across axis 210. Pin 234 passes through and “captures” or locks the struts in place as they are bent, thus preventing the struts from snapping back to their outward circumferential position. As struts 232 are bent inward and captured by pin 234, ribs 230 are drawn inward as well, as shown in FIG. 35B. By passing pin 234 along the entire length of axis 210 through implant 228, the implant is brought into the closed configuration, wherein its outer diameter is substantially reduced. Preferably the diameter or dimension of the implant in the closed configuration of FIG. 35B is reduced to at least half the diameter in the open configuration shown in FIG. 35A.

Once implant 228 has been inserted into the sinus tarsi of a foot, pin 234 is removed. Upon removal of the pin, struts 232 spring back to their original, circumferential positions, and the implant resumes the open configuration shown in FIG. 35A.

As described above, implant 228 may, if desired, be made of shape memory material, which in its normal, austenitic state maintains the open configuration with substantial rigidity. As struts 232 are bent, they assume a state of stress-induced martensite, returning to the austenitic state when the stress is removed as pin 234 is removed. If desired, this implant can be covered with a sheath or sleeve (such as an expandable flexible polymer) to prevent bone ingrowth.

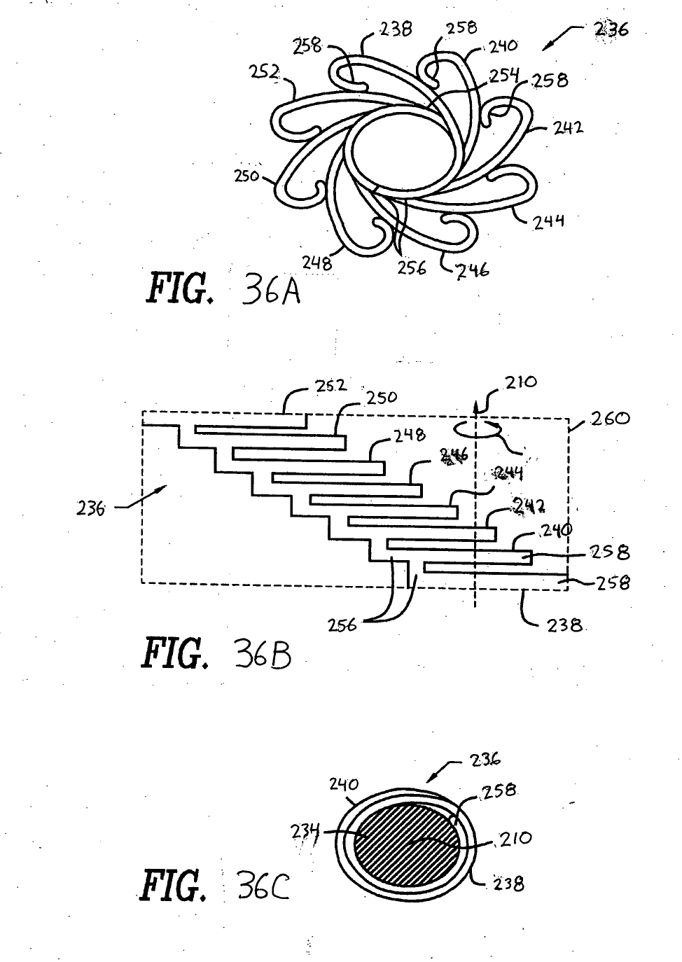

As a further embodiment to those described above, another self-expandable subtalar implant is shown in FIGS. 36A through 36C. The preferred material for this implant is Nitinol, although the device can also be made from a polymer, stress-induced martensite (SIM), smooth tin, or other suitable materials.

In accordance with another embodiment of the present invention, FIG. 36A is a schematic, end view of this self-expanding subtalar implant 236 in an open configuration. Implant 236 is preferably formed of resilient material, more preferably superelastic material, as described above. The implant comprises a plurality of leaves 238, 240, 242, 244, 246, 248, 250 and 252, extending radially outward in a spiral pattern about axis 210 of the implant, the leaves extending from a central, generally tubular portion 254. As shown in FIG. 36A, each of the leaves extends outward at a different angle about axis 210 (as measured off of a single reference line, not shown, extending from the axis to a point located at 0 degrees on the circumference). In the expanded configuration of FIG. 36A, the leaves engage the inner surface of the sinus tarsi of a foot in order to hold implant 236 in place and alter subtalar joint motion. Each of the leaves has a base 256, which forms a part of tubular portion 254 of the implant, and an inward-curved end portion 258.

FIG. 36B is a schematic illustration showing a flat sheet of resilient material 260, which is cut in preparation for fabrication of implant 236. Leaves 238, 240, 242, 244, 246, 248, 250 and 252 are cut out of sheet 260 in a stairstep pattern, i.e. each leaf presents a step-like extension, as shown in FIG. 36B. The leaves are then rolled up, one after the other. The leaves are rolled about axis 210, in the direction indicated by arrow 262, so that in the closed configuration shown in FIG. 36C, the leaves will expand to the shape shown in FIG. 36A.

FIG. 36C is a schematic, sectional illustration showing implant 236 in the closed configuration, in preparation for insertion of the implant into the sinus tarsi. Holding pin 234, as described above with reference to FIG. 35B, is inserted along axis 210 of implant 236. Curved end portions 258 of leaves 238, 240, 242, 244, 246, 248, 250 and 252 are bent inward and hooked around pin 234. Implant 236 remains in this closed configuration as long as pin 234 is in place. In the closed configuration, the device maintains a smaller external diameter than the open configuration, to facilitate insertion of the device into the sinus tarsi. After insertion of the implant in the sinus tarsi, pin 234 is withdrawn, and the resilience of the leaves causes them to spring outward, so that implant 236 resumes the open, larger diameter, configuration shown in FIG. 36A. In this larger diameter, support of the subtalar joint is provided as previously described above.

The implant, as with the other devices in the application, can also expand by heating, taking advantage of the material's shape memory properties. As with the other embodiments of the invention disclosed herein, it can be used in treatment of both the subtalar joint and other foot joints, including but not limited to the 1st MTP joint.

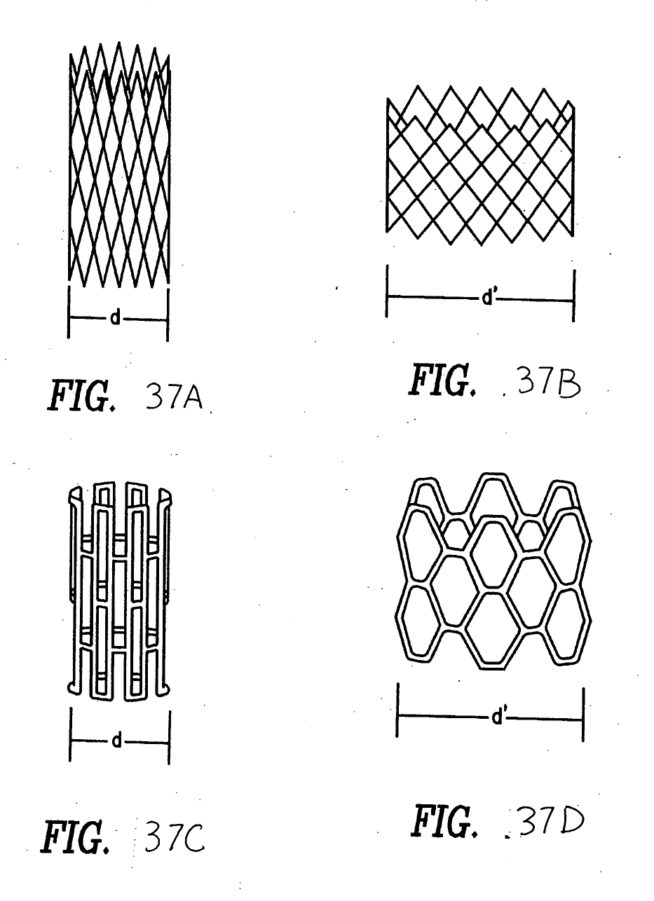

As an alternative to a folded construction, the expandable subtalar implant can be configured based on a lattice configuration. Representative embodiments are shown in FIGS. 37A through 37D, which illustrates a series of perspective views of two embodiments of the configuration in both the small, constricted, diameter and the large, expanded, diameter. These embodiments can be inserted into the foot, taking advantage of the self-expanding principle inherent to superelastic or shape memory alloys discussed above. Alternatively or additionally, the implants may be balloon expanded or further supported by an inflatable, detachable balloon as previously described.

In the preferred embodiments of FIGS. 37A through 37D, the implants are each formed in a meshwork or lattice configuration. FIGS. 37A and 37B provide an illustration of a one embodiment of this lattice configuration, while FIGS. 37C and 37D provide an illustration of another embodiment. As shown in FIGS. 37A and 37C, a first, small profile state is illustrated for each of the implants in which the implants are compressed into small diameters d. This reduced diameter facilitates ease of insertion into the sinus tarsi. FIGS. 37B and 37D show the respective embodiments, each with increased diameters d′ after expansion. After insertion into sinus tarsi, the implants may be transformed to their enlarged, implantation configuration such as by inflation of an expansion balloon, or due to the properties of the superelastic or shape memory material.

Although of similar construction, these first and second embodiments differ in the design of their respective lattices. One embodiment (FIGS. 37A and 37B) is constructed as a lattice which is initially in a configuration that is substantially diamond shaped, and which expands outward into a series of expanded diamonds or squares. Another embodiment is constructed as a reduced-size lattice having a series of rectangular shaped subunits, which expand outward to form a series of interconnected hexagons (six-sided polygons), like a honeycomb. Suitable lattice structures may be formed such as by laser etching from tube stock, or by weaving or other fabrication from wire or ribbon of a suitable material.

In addition to the embodiments shown, other meshworks or lattices may also be provided. Likewise, although the embodiments shown are preferably for use in self-expanding designs, they can be constructed out of other materials to serve as expandable implants. Such expandable devices, as disclosed below, will expand from the reduced to the expanded diameter state upon application of suitable energy or force.

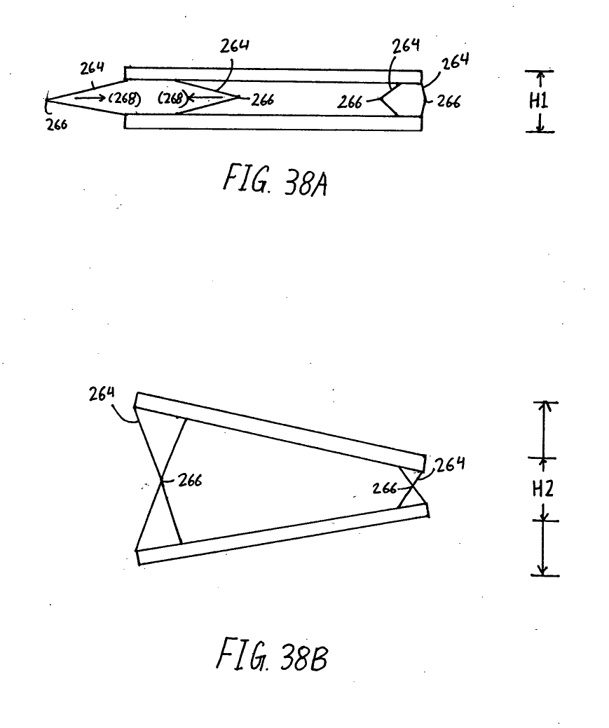

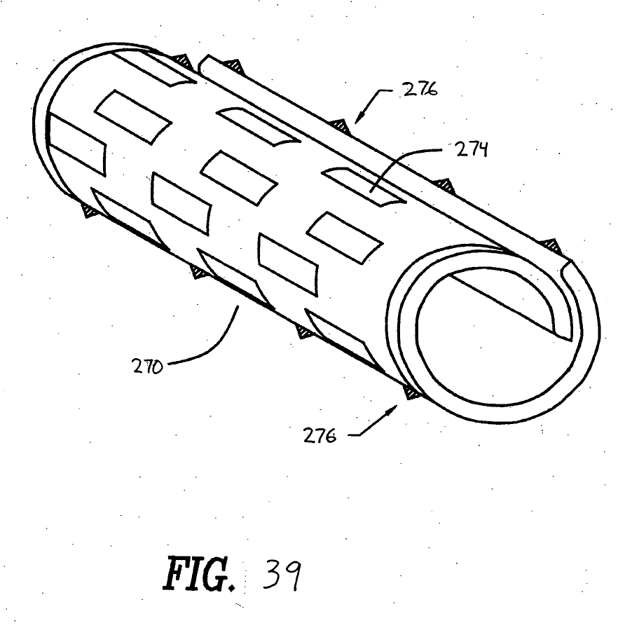

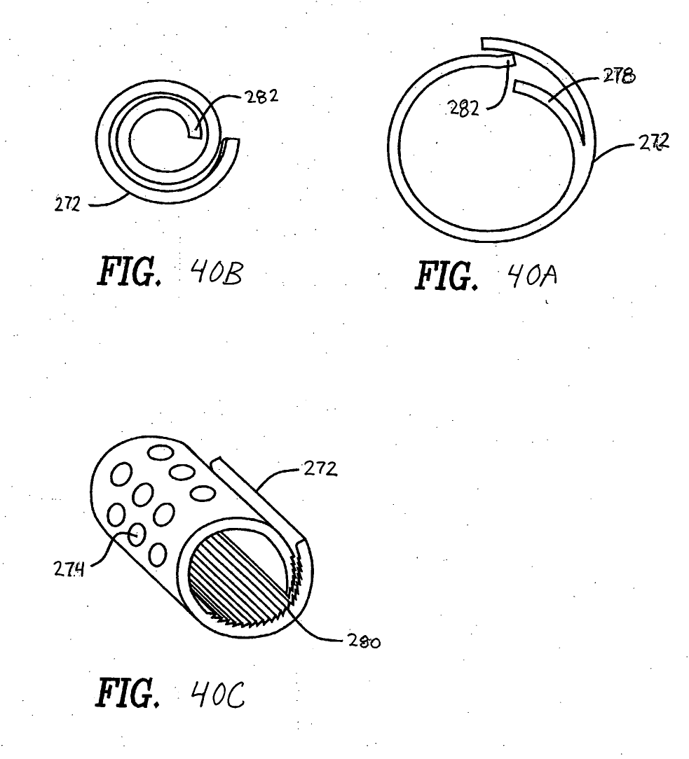

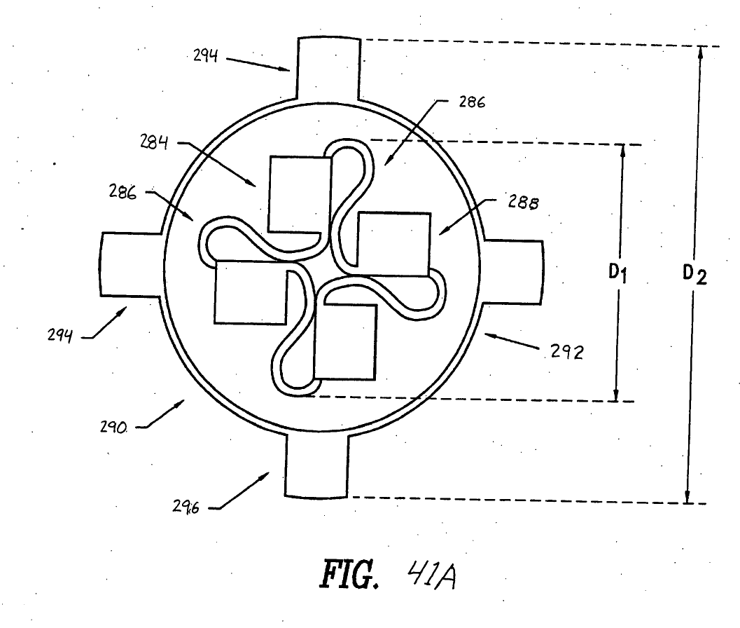

FIGS. 38A and 38B illustrate a further embodiment of the invention. The implant is constructed as a frusta-conical device which can be set to two heights, H1 and H2. Rigid rods or bars 264 are hinged at points 266. By applying external force 268 on the hinge 266, the height of the implant can be changed, thereby providing its expansion and fixation properties at its new height, H2 (compare FIG. 38B to FIG. 38A).

Although preferred embodiments are described herein with reference to arthroereisis of the subtalar joint, other embodiments of the invention provide use of an expandable implant in other joints of the foot, including but not limited to joints such as an MTP joint.

The implants and minimally-invasive methods of accessing the sinus tarsi in accordance with the present invention, appropriately adapted for the anatomical features of the other foot joints being treated, have the advantages of minimizing operative trauma and damage to soft tissues. Furthermore, the patient is able to mobilize the treated foot more quickly than the prior art.

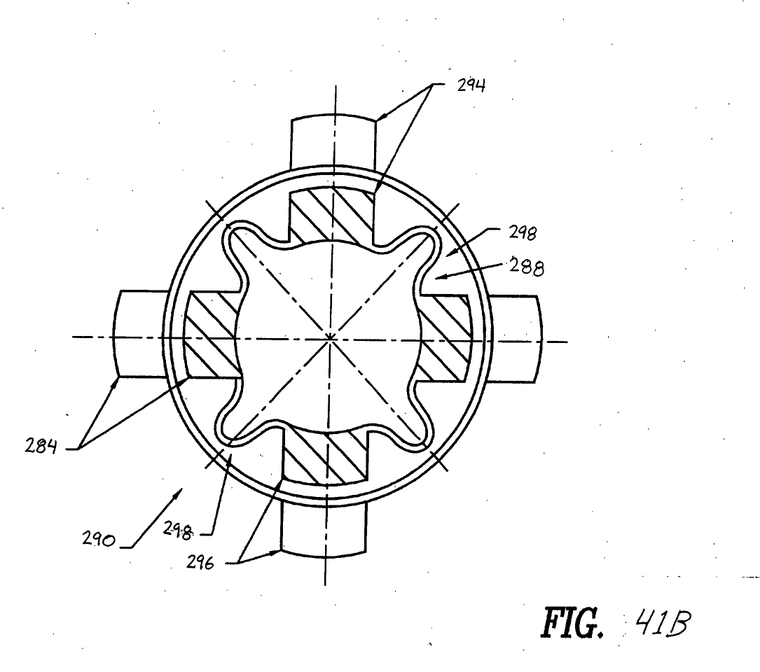

As shown in FIGS. 39 and 40A through 40C, embodiments of the subtalar implant are illustrated. The subtalar implant 270 or 272 is initially inserted through the syringe or catheter in the compressed, reduced diameter form illustrated in FIG. 40B. This implant is initially maintained in a reduced diameter profile for insertion into the sinus tarsi. This ability to percutaneously insert the implant, due to the reduced diameter profile of the implant, allows major surgery to be avoided and reduces the trauma and risk of infection to the patient.

Upon insertion of the implant 270 or 272 into the sinus tarsi, the implant uncoils to reach the expanded state shown in FIG. 40C, by virtue of its expandable properties. As with the embodiments of the inventions disclosed above, the implant 270 or 272 is preferably made of biocompatible metal or polymer and is initially inserted through the syringe or catheter in the compressed, reduced diameter form illustrated in FIG. 40C. The implant can also be made of materials such as annealed 316-L stainless steel, shape memory alloy (e.g. Nitinol), or a polymer such as polyurethane. In the event that annealed material is used, the implant 270 or 272 will require the assistance of an expander to expand its diameter after insertion. This expander can be a balloon inserted through the syringe or catheter which is inflated to dilate the implant to the diameter of the sinus tarsi. Alternatively, the expander can be a mechanical expander which is inserted into implant lumen and which self-expands, or which is expanded using outside assistance. In the event that a self expandable material is used for the implant, this implant can still be employed merely to assist with the expansion, if desired or needed. Alternatively or additionally, the implants may be balloon expanded or further supported by an inflatable, detachable balloon as previously described.

As can be seen with reference to FIG. 39 or 40C, the implant 270 or 272 is provided with a series of pores or gaps 274 in its surface. These pores 274 (which are circular, rectangular, or any other shape) enhance anchoring ability of the implant by allowing bone growth through the pores while the spacer is in place. Protrusions or spikes 276 can also be provided, which penetrate the bone surface and assist with anchoring of the implant.

As further shown in FIGS. 40A and 40C, in the preferred embodiments, implant 272 is provided with a locking mechanism such as one or more locking fingers 278 or teeth 280. This locking mechanism further maintains the expanded diameter of the implant 272 and retards or prevents compression of the implant 272 back to its reduced diameter state. FIG. 40A illustrates the use of one or more locking fingers 278 in implant 280. When implant 272 expands, leading edge 282 of the implant travels past and over locking fingers 278 or teeth 280. Locking fingers 278 or teeth 280 resist retrograde movement of the leading edge 282 or contraction of the implant 272 by trapping the leading edge 282 within the “V” shaped gap of the locking finger 278, or the groove of one of the teeth 280. As a result, in response to the application of force to implant 272 while it rests in the sinus tarsi, the implant exhibits flexible compressive characteristics yet resists undue compression, due to the counteraction provided by the locking mechanism.

Another embodiment of the subtalar joint implant of the invention is provided in FIG. 41A. FIG. 41A depicts two cross sectional views of an implant, both before and after expansion, these views being superimposed on each other (for appreciation of relative constricted and expanded diameters). In one embodiment of the invention, the constricted implant includes a curved or undulated surface, preferably having longitudinal bars 284 located thereon. It is preferred that the implant, before expansion, have its surface be curved or folded inward to form a series of connected bulbous sections 286. In the preferred embodiment, the bulbous sections form a clover like configuration in the compressed state, as shown in the four leaf clover configuration illustrated in FIG. 41A.

As shown in FIG. 41A, in the compressed configuration or state 288, implant 290 maintains a compressed diameter D1. Compressed diameter D1 is a small diameter such that the implant is suitable for insertion into the sinus tarsi through an incision or puncture site in the foot. In contrast, in the expanded configuration or state 334, the implant 290 is maintained within the bone at an expanded diameter D2. Expanded diameter D2 is a larger diameter, measured from the outside surface of longitudinal bar 294 to the outside surface of opposing longitudinal bar 296, this diameter being sufficient such that the longitudinal bars are pressed up against the inner wall of the sinus tarsi. FIG. 41A, although not to scale, shows both the compressed and expanded states of the implant superimposed on each other, illustrating the substantial increase of diameter achieved by enlargement of the implant from the compressed to the expanded state.

FIG. 41B is a further embodiment of the invention, illustrated in the same manner as in FIG. 41A. In this embodiment, one or more hairpin loops or arcs 298 are provided between longitudinal bars 284. In the embodiment shown, four longitudinal bars 284 are provided, each at 90 degrees to each other, with one hairpin loop 298 centrally located between and connecting each adjacent pair of longitudinal bars. One or more, or no hairpin loops, can be provided between any or all of the pairs of adjacent longitudinal bars, if desired.

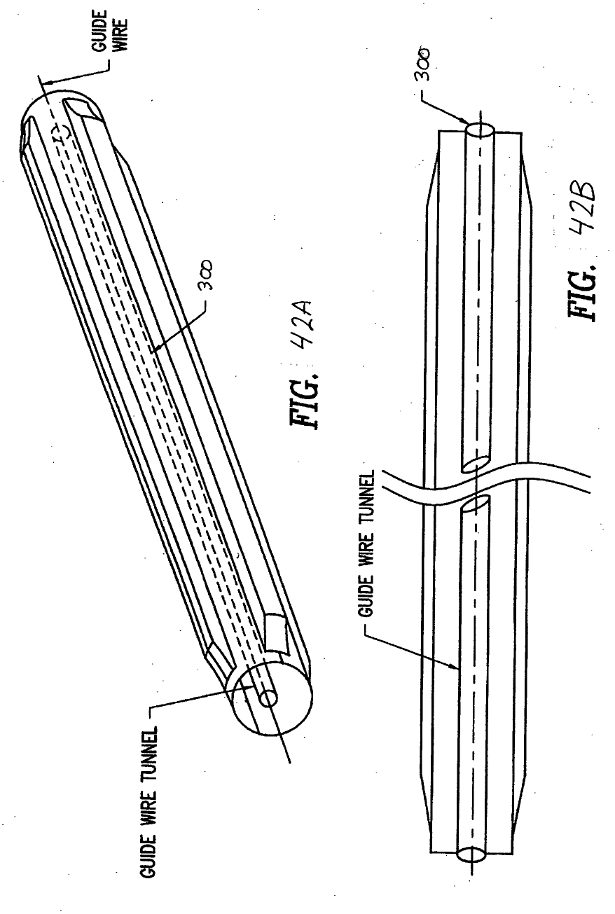

As shown in FIGS. 42A and 42B, in preferred embodiments, the implant can also be provided with a medial longitudinal canal, bore or tunnel 300. This canal 300 facilitates the insertion of the implant into the foot, allowing the insertion procedure to be performed using a guidewire. The medial canal 300 is threaded over the guidewire to allow the implant to be easily guided into the appropriate position during insertion into the sinus tarsi, and to allow the guidewire to be pulled out once positioning has been completed.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

1. A method for treating a patient, comprising the steps of:
providing a self-expandable subtalar implant;
inserting said implant into the sinus tarsi of a foot; and
allowing self-expansion of said implant in the sinus tarsi.

2. The method of claim 1, further comprising the step of changing the alignment of the hindfoot.

3. The method of claim 1, wherein said inserting step is performed through a cannula inserted into said sinus tarsi of said patient.

4. The method of claim 1, wherein said inserting step is performed over a guidewire inserted into said sinus tarsi of said patient.

5. The method of claim 1, further comprising the steps of:
inserting a balloon catheter in said implant; and
expanding the balloon of said catheter.

6. The method of claim 5, further comprising the step of;
detaching said balloon from said catheter.

7. A method for treating a patient, comprising the steps of:
providing a self-expanding subtalar implant;
identifying a foot having a first range of motion;
inserting said implant into the sinus tarsi of said foot; and
adapting said foot to a second range of motion by allowing self-expansion of said implant.

8. A method for treating a patient, comprising the steps of:
providing a self-expandable subtalar implant;
identifying a foot having a first weight-bearing alignment;
limiting said foot to a second weight-bearing alignment;
inserting said implant into a sinus tarsi of a foot; and
securing said foot in said second weight-bearing alignment by allowing self-expansion of said implant.

9. The method of claim 8, wherein said first and second weight-bearing alignments are defined by the angle between a first line connecting the edges of an articular surface of a talus and a second line connecting the edges of an articular surface of a navicular bone.

10. A method for treating a patient, comprising the steps of:
providing an expandable subtalar implant with an internal lumen;
inserting said implant into the sinus tarsi of a foot; and
expanding said implant by plastic deformation of at least a portion of said implant.

11. The method of claim 10, wherein the expandable subtalar implant of the providing step has a first end, a second end and a middle deformable portion that is capable of radial expansion by moving the first end and second end in closer proximity.

12. The method of claim 11, wherein the expanding step comprises moving the first end and the second end of said implant in close proximity.

13. The method of claim 10, further comprising the step of changing the alignment of the hindfoot.

14. The method of claim 10, wherein said inserting step is performed through a cannula inserted into said sinus tarsi of said patient.

15. The method of claim 10, wherein said inserting step is performed over a guidewire inserted into said sinus tarsi of said patient.

16. The method of claim 10, wherein said expanding step is performed by a balloon catheter.

17. A method for treating a patient, comprising the steps of:
providing an expandable subtalar implant;
identifying a foot having a first range of motion;
inserting said implant into the sinus tarsi of said foot; and
adapting said foot to a second range of motion by deformably expanding said implant.

18. A method for treating a patient, comprising the steps of:
providing an expandable subtalar implant;
identifying a foot having a first weight-bearing alignment;
limiting said foot to a second weight-bearing alignment;
inserting said implant into a sinus tarsi of a foot; and
securing said foot in said second weight-bearing alignment by deforming expansion of said implant.

19. The method of claim 18, wherein said first and second weight-bearing alignments are defined by the angle between a first line connecting the edges of an articular surface of a talus and a second line connecting the edges of an articular surface of a navicular bone.

20. The method of claim 18, wherein said first and second weight-bearing alignments are defined by the angle between a first line along the long axis of a talus and a second line along the long axis of a first metatarsal bone.

21. The method of claim 18, wherein said first and second weight-bearing alignments are defined by the angle between a first line between the plantar-most point of a calcaneus of a patient and a most plantar point of the distal articular surface of said calcaneus, and a second line within a horizontal plane of said patient.

22. The method of claim 18, wherein said first and second weight-bearing alignments are defined by the angle between a first line along the plantar border of a calcaneus and a second line along a first midpoint in the body of a talus and a second midpoint in the neck of said talus.

23. A method for treating a patient, comprising the steps of:
identifying a cyma line in a foot of a patient;
smoothing said cyma line; and
securing said smoothing by expanding an implant in the sinus tarsi of said foot.

24. A method of treating a patient, comprising the steps of:
accessing a sinus tarsi of a foot through an access path having a cross sectional diameter of no more than about 0.5 inches, the sinus tarsi having a talus and calcaneus spaced apart by a first minimum distance;
increasing the space between the talus and calcaneus to a second minimum distance; and
restraining the talus and calcaneus at said second minimum distance.

25. A method for treating a patient, comprising the steps of:
providing an expandable first metatarsal-phalangeal joint implant;
inserting said implant into a first metatarsal-phalangeal joint of a foot; and
expanding said implant with a fluid.

26. A method for treating a patient, comprising the steps of:
providing an mass-increasable subtalar implant;
inserting said implant into the sinus tarsi of a foot; and
allowing self-expansion of said implant in the sinus tarsi.

27. The method of claim 26, further comprising the step of changing the alignment of the hindfoot.

28. The method of claim 26, wherein said inserting step is performed through a cannula inserted into said sinus tarsi of said patient.

29. The method of claim 26, wherein said inserting step is performed over a guidewire inserted into said sinus tarsi of said patient.

30. The method of claim 26, further comprising the steps of:
inserting a balloon catheter in said implant; and
expanding the balloon of said catheter.

31. The method of claim 30, further comprising the step of detaching said balloon from said catheter.

32. A method for treating a patient, comprising the steps of:
providing a mass-increasable subtalar implant;
identifying a foot having a first range of motion;
inserting said implant into the sinus tarsi of said foot; and
adapting said foot to a second range of motion by increasing the mass of said implant.

33. A method for treating a patient, comprising the steps of:
providing a mass-increasable subtalar implant;
identifying a foot having a first weight-bearing alignment;
limiting said foot to a second weight-bearing alignment;
inserting said implant into a sinus tarsi of a foot; and
securing said foot in said second weight-bearing alignment by increasing the mass of said implant.

34. The method of claim 33, wherein said first and second weight-bearing alignments are defined by the angle between a first line connecting the edges of an articular surface of a talus and a second line connecting the edges of an articular surface of a navicular bone.

35. A method for treating a patient, comprising the steps of:
providing an inflatable subtalar implant;
inserting said implant into the sinus tarsi of a foot; and
inflating said implant with an inflation material.

36. The method of claim 35, wherein said material is a fluid.

37. The method of claim 35, wherein said material is a solid.

38. The method of claim 37, wherein said solid comprises microspheres.

39. The method of claim 35, further comprising the step of changing the alignment of the hindfoot.

40. The method of claim 35, wherein said inserting step is performed through a cannula inserted into said sinus tarsi of said patient.

41. The method of claim 35, wherein said inserting step is performed over a guidewire inserted into said sinus tarsi of said patient.

42. The method of claim 35, further comprising the step of combining multiple agents to form said inflation material.

43. The method of claim 42, wherein said combining step is performed before said inflating step.

44. The method of claim 42, wherein said combining step is performed during said inflating step.

45. A method for treating a patient, comprising the steps of:
providing an inflatable subtalar implant;
identifying a foot having a first range of motion;
inserting said implant into the sinus tarsi of said foot; and
adapting said foot to a second range of motion by inflating said implant.

46. A method for treating a patient, comprising the steps of:
providing an inflatable subtalar implant;
identifying a foot having a first weight-bearing alignment;
limiting said foot to a second weight-bearing alignment;
inserting said implant into a sinus tarsi of a foot; and
securing said foot in said second weight-bearing alignment by inflating said implant.

47. The method of claim 46, wherein said first and second weight-bearing alignments are defined by the angle between a first line connecting the edges of an articular surface of a talus and a second line connecting the edges of an articular surface of a navicular bone.

48. The method of claim 46, wherein said first and second weight-bearing alignments are defined by the angle between a first line along the long axis of a talus and a second line along the long axis of a first metatarsal bone.

49. The method of claim 46, wherein said first and second weight-bearing alignments are defined by the angle between a first line between the plantar-most point of a calcaneus of a patient and an most inferior point of the distal articular surface of said calcaneus, and a second line within a horizontal plane of said patient.

50. The method of claim 46, wherein said first and second weight-bearing alignments are defined by the angle between a first line along the plantar border of a calcaneus and a second line along a first midpoint in the body of a talus and a second midpoint in the neck of said talus.

51. A minimally invasive method for treating a patient, comprising the steps of:
providing an inflatable subtalar implant;
inserting said implant into a sinus tarsi of a foot;
inflating said implant;
changing the range of motion of the subtalar joint of said foot; and
conforming the implant to the shape of the sinus tarsi thereby.

52. A method for treating a patient, comprising the steps of:
identifying a cyma line in a foot of a patient;
smoothing said cyma line; and
securing said smoothing by inflating an implant in the sinus tarsi of said foot.

53. A method of treating a patient, comprising the steps of:
accessing a sinus tarsi of a foot through an access path having a cross sectional diameter of no more than about 0.5 inches, the sinus tarsi having a talus and calcaneus spaced apart by a first minimum distance;
increasing the space between the talus and calcaneus to a second minimum distance; and
restraining the talus and calcaneus at said second minimum distance.

54. A method for treating a patient, comprising the steps of:
providing an inflatable first metatarsal-phalangeal joint implant;
inserting said implant into a first metatarsal-phalangeal joint of a foot; and
inflating said implant with a fluid.

55. A subtalar joint implant, comprising:
an inflatable balloon adapted for positioning in the sinus tarsi of a foot.

56. A foot implant, comprising:
an inflatable balloon, wherein said inflatable balloon is adapted for extra-articular positioning in the sinus tarsi of the foot.
